(19) United States
(12) Patent Application Publication
(10) Pub. No.: US 2017/0079658 A1
(43) Pub. Date: Mar. 23, 2017

(54) INSITU SWAPPABLE CLIP DELIVERY CARTRIDGE

(71) Applicant: Empire Technology Development LLC, Wilmington, DE (US)

(72) Inventor: Worth Walters, Los Gatos, CA (US)

(21) Appl. No.: 14/858,947

(22) Filed: Sep. 18, 2015

(51) Int. Cl. A61B 17/10; A61B 17/08

(52) U.S. Cl. CPC A61B 17/105; A61B 17/083; A61B 17/10

(57) ABSTRACT

Several embodiments disclosed herein relate to apparatus and methods for treating a diverticulum. In some examples, disclosed is a clip placement device for diverticulum inversion. The clip placement device can include a tubular body, a clip attachment structure, and a pusher structure. In some examples, the clip attachment structure has a distal end that is attachable to a clip and wherein the clip attachment structure is configured to engage with the distal end of the tubular body. In some examples, the pusher structure is configured to removeably connect to the clip attachment structure.

### BACKGROUND

An outpouching of the colon or other body lumen, called a diverticulum, can become the site for inflammation known as diverticulitis, microperforation and/or bleeding. Current treatments may involve the surgical removal of segments of the body lumen. For extreme cases of diverticulitis, treatment can involve colon resection and placement of a colostomy. This approach results in significant healthcare costs and substantial pain for patients.

### SUMMARY

Disclosed is a device for inverting a diverticulum. In some embodiments, disclosed is a clip placement device for diverticulum inversion including a tubular body, a clip attachment structure, and a pusher structure. In some examples, clip attachment structure has a distal end that is attachable to a clip. In some examples, the clip attachment structure is further configured to engage with the distal end of the tubular body. In some examples, the pusher structure is coaxially disposed within the tubular body. In some examples, the pusher structure is configured to removeably connect to the clip attachment structure.

In some embodiments, disclosed is a method of clip placement for diverticulum inversion. In some examples, the method can include positioning a distal end of a clip placement device along an outer wall of a colon at a diverticulum. In some examples, the method can further include inverting the diverticulum into the lumen of the colon with the distal end of the clip placement device, wherein the clip placement device comprises a tubular body and a pusher structure that is coaxially disposed within the tubular body, and wherein the pusher structure is configured to attach to a clip attachment structure. In some examples, the method can include attaching the clip attachment structure to the pusher structure, wherein the clip attachment structure has a distal end that is attached to a clip. In some examples, the method can further include advancing the pusher structure and the attached clip attachment structure along the tubular body until the clip protrudes from the distal end of the tubular body.

In other embodiments, the method can further include withdrawing the pusher structure with the attached clip attachment structure such that the pusher structure is withdrawn from the tubular body. In some examples, the method can further include removing the attached clip attachment structure from the pusher structure. In some examples, the method can further include attaching a second clip attachment structure to the pusher structure, wherein the second clip attachment structure has a distal end that is attached to a second clip. In some examples, the method can include advancing the pusher structure and the attached second clip attachment structure along the tubular body until the second clip protrudes from the distal end of the tubular body.

In other embodiments, the method can include a clip attachment structure that is configured to engage with the distal end of the tubular body.

In some embodiments, disclosed is a system for clip placement and diverticulum inversion that includes a clip placement device. In some examples, the clip placement device can include a tubular body, a clip attachment structure, and a pusher structure. In some examples the clip attachment structure can have a distal end that is attached to a clip, and wherein the clip attachment structure is configured to engage with the distal end of the tubular body. In some examples, the pusher structure is coaxially disposed within the tubular body, wherein the pusher structure is configured to removeably connect to the clip attachment structure. In some examples, the system for clip placement and diverticulum inversion further includes a second clip attachment structure, wherein the second clip attachment structure has a distal end that is attached to a second clip, and wherein the second clip attachment structure is configured to engage with the distal end of the tubular body and is configured to removeably connect to the pusher structure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A illustrates a side view of an embodiment of a device for inverting diverticulum with all of the components of the device exposed.

FIG. 1B illustrates a side view of the device illustrated in FIG. 1A with a few of the components retracted.

FIGS. 1C-K illustrate a plurality of views of a method for treating diverticulum disease using the device of FIG. 1A.

FIG. 1L illustrates a flowchart of an embodiment of the method for treating diverticulum disease illustrated in FIGS. 1C-K.

FIG. 2A illustrates a side view of another embodiment of a device for inverting diverticulum.

FIGS. 2B-H illustrate a plurality of views of a method for treating diverticulum disease using the device of FIG. 2A.

FIG. 3A illustrates a side view of another embodiment of a device for inverting diverticulum.

FIG. 3B illustrates a top view of an embodiment of a closure clip in a relaxed configuration wherein the closure clip can be used with the devices illustrated in FIGS. 1A, 2A, and 3A.

FIG. 3C illustrates a side view of the closure clip of FIG. 3B when it is loaded on any of the devices illustrated in FIGS. 1A, 2A, and 3A.

FIG. 3D illustrates a side view of the closure clip of FIG. 3B as it is delivered to a target site.

FIG. 3E illustrates a side view of an embodiment of a clip tube of the device of FIG. 3A.

FIG. 3F illustrates the clip tube of FIG. 3E that is loaded with the closure clip illustrated in FIG. 3D.

FIGS. 3G-I illustrate a plurality of views of the distal end of an embodiment of a push rod of the device of FIG. 3A.

FIG. 3J illustrates a side view of the clip tube of FIG. 3E and push rod of FIGS. 3G-I that is loaded with the closure clip illustrated in FIG. 3D.

FIG. 3K illustrates a side view of the apparatus of 3J where the closure clip is configured for delivery as illustrated in FIG. 3E.

FIG. 3L illustrates an embodiment of a sheath of the device of FIG. 3A.

FIGS. 3M-S illustrate a plurality of views of a method for treating diverticulum using the device of FIG. 3A.

FIG. 4 illustrates an exploded view of an embodiment of a device with swappable cartridges for inverting diverticulum.

FIG. 5A illustrates a side view of an embodiment of a device with swappable cartridges for inverting diverticulum.

FIGS. 5B-D illustrate a plurality of views of the handle of the device of FIG. 5A.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

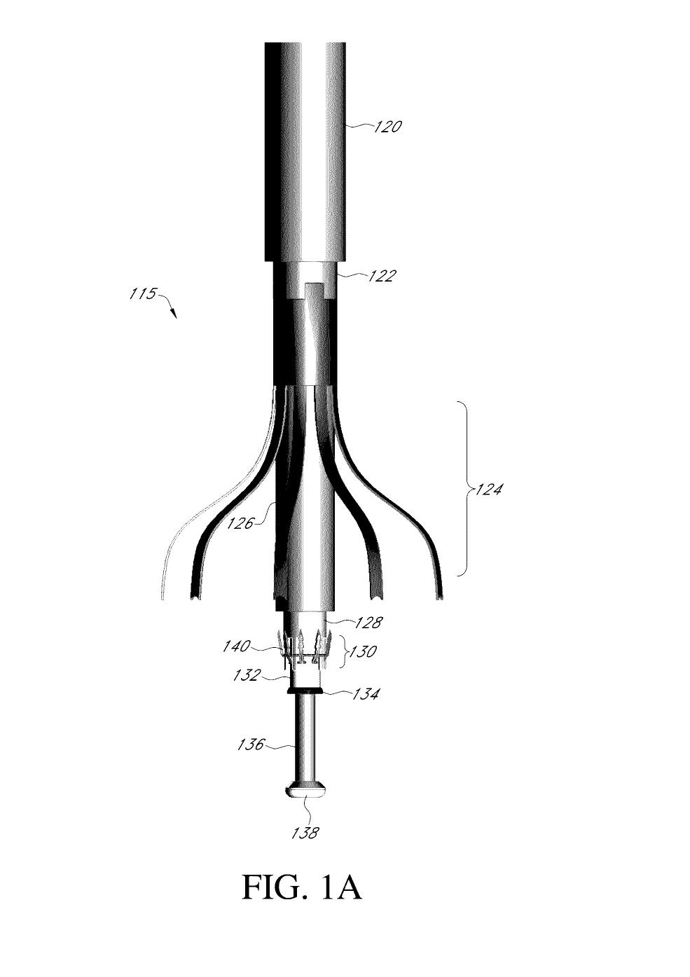

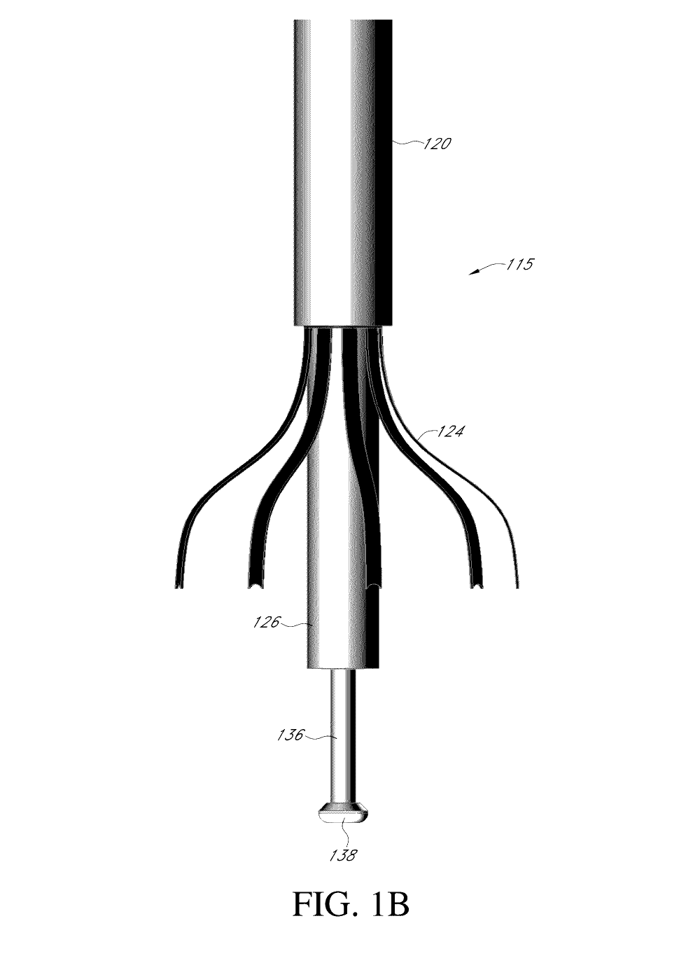

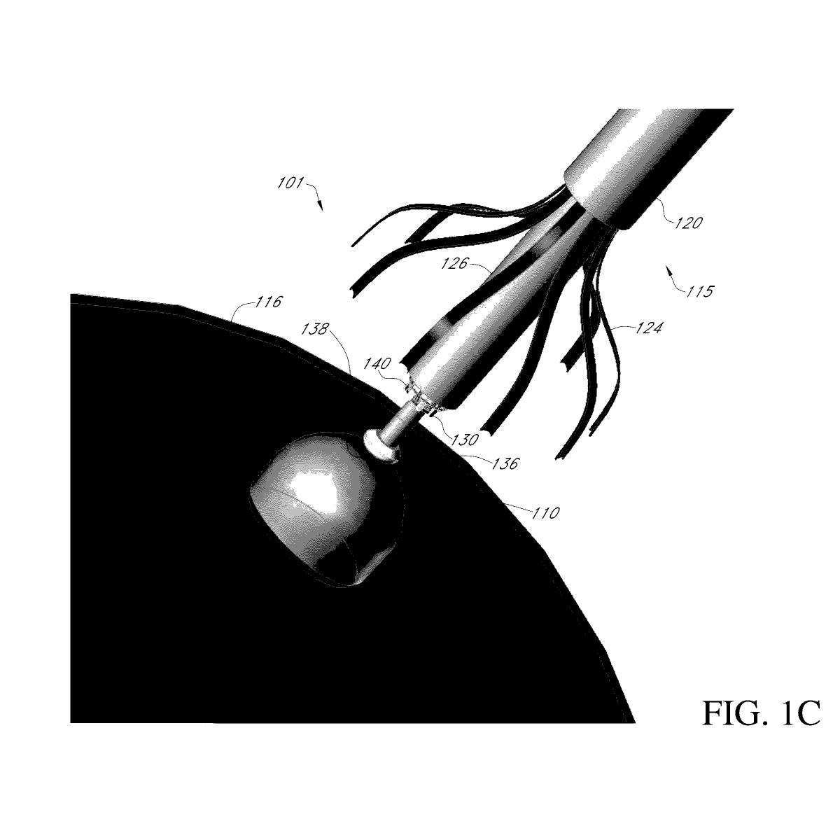

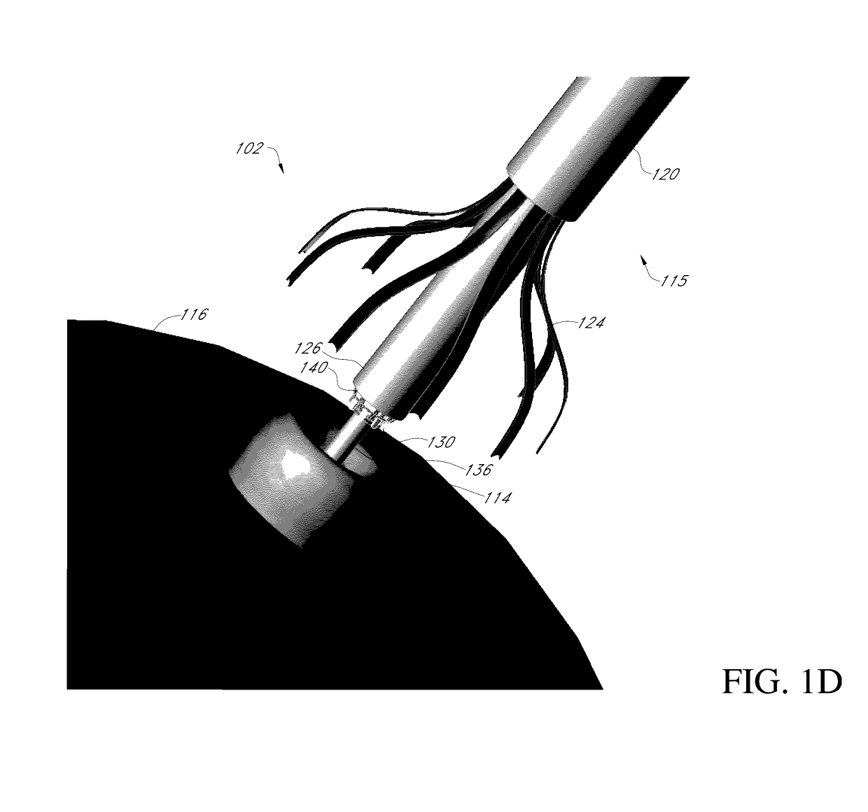

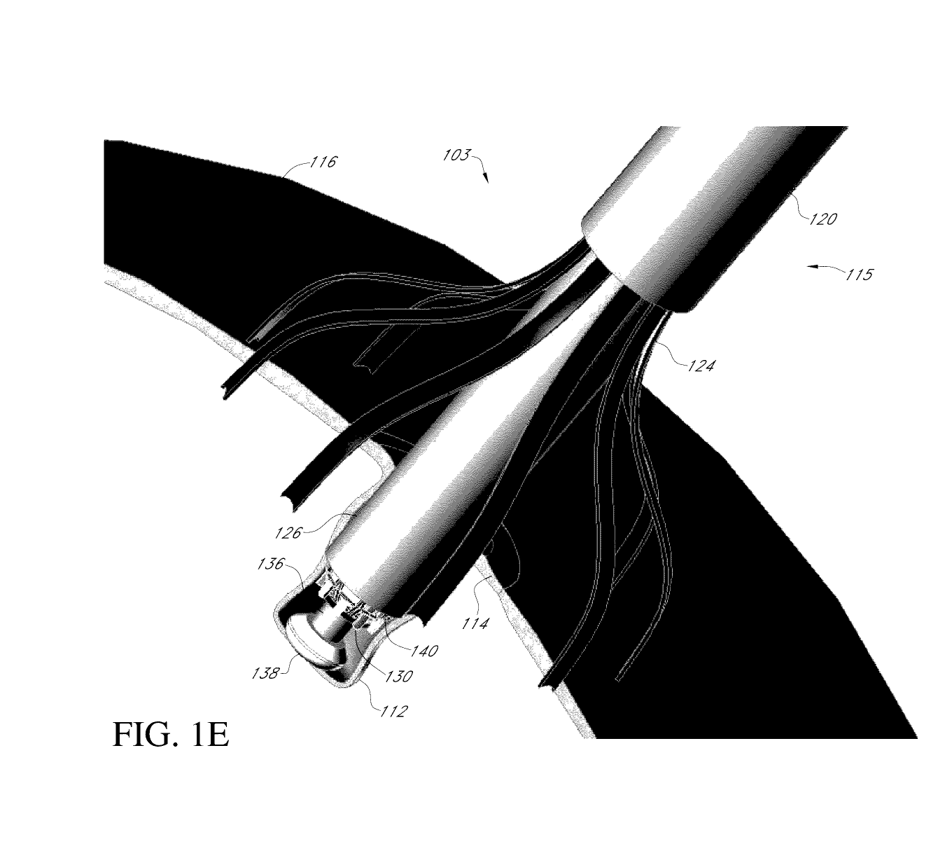

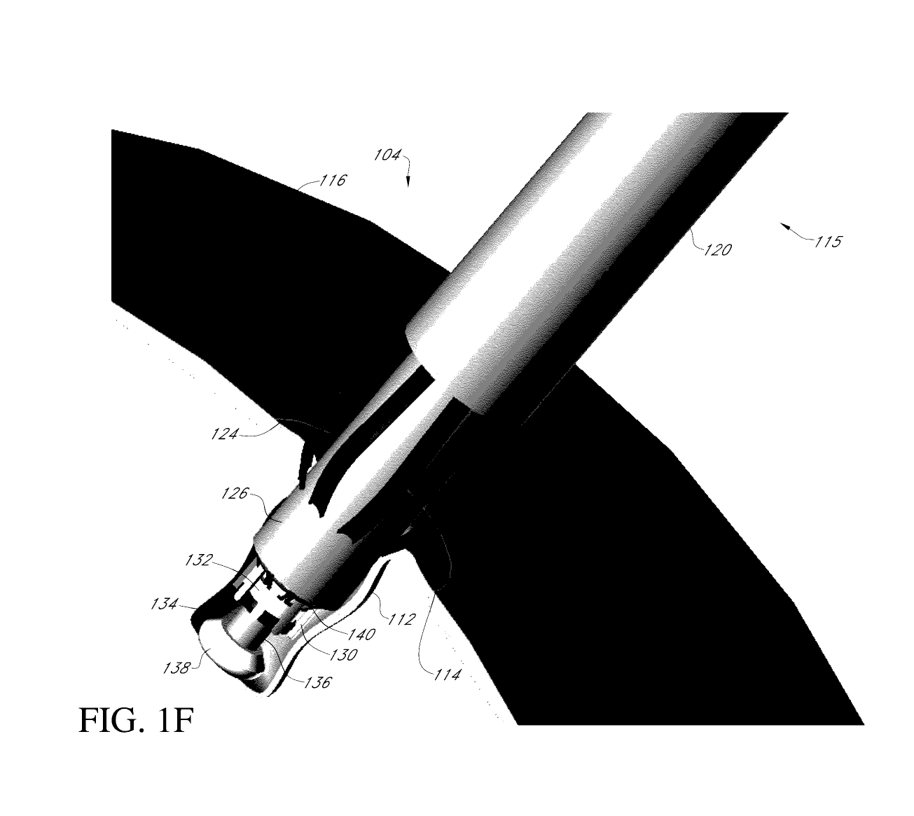

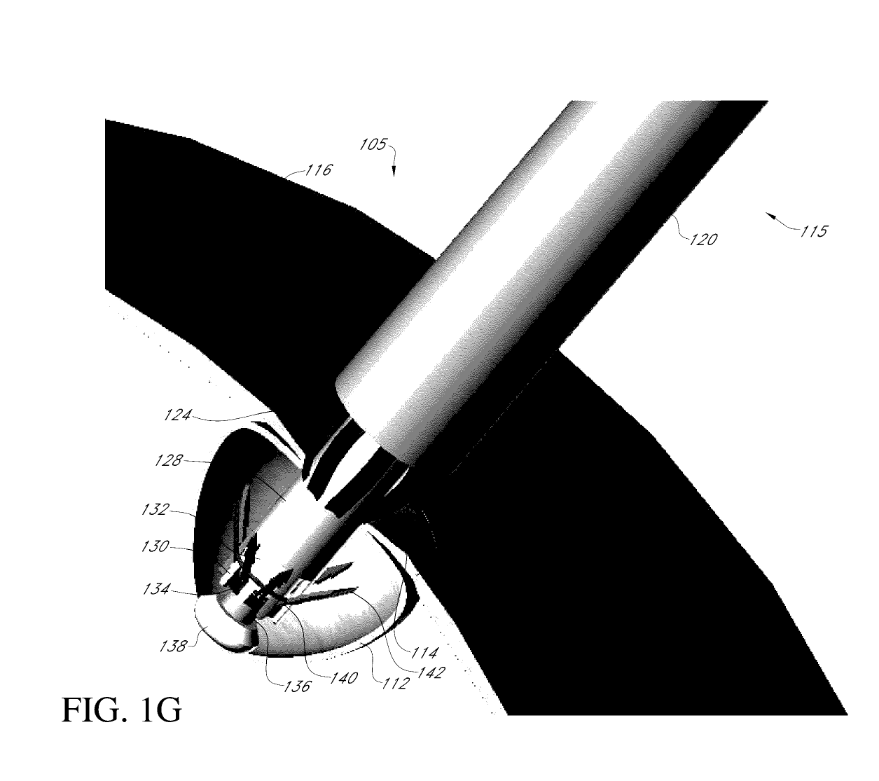

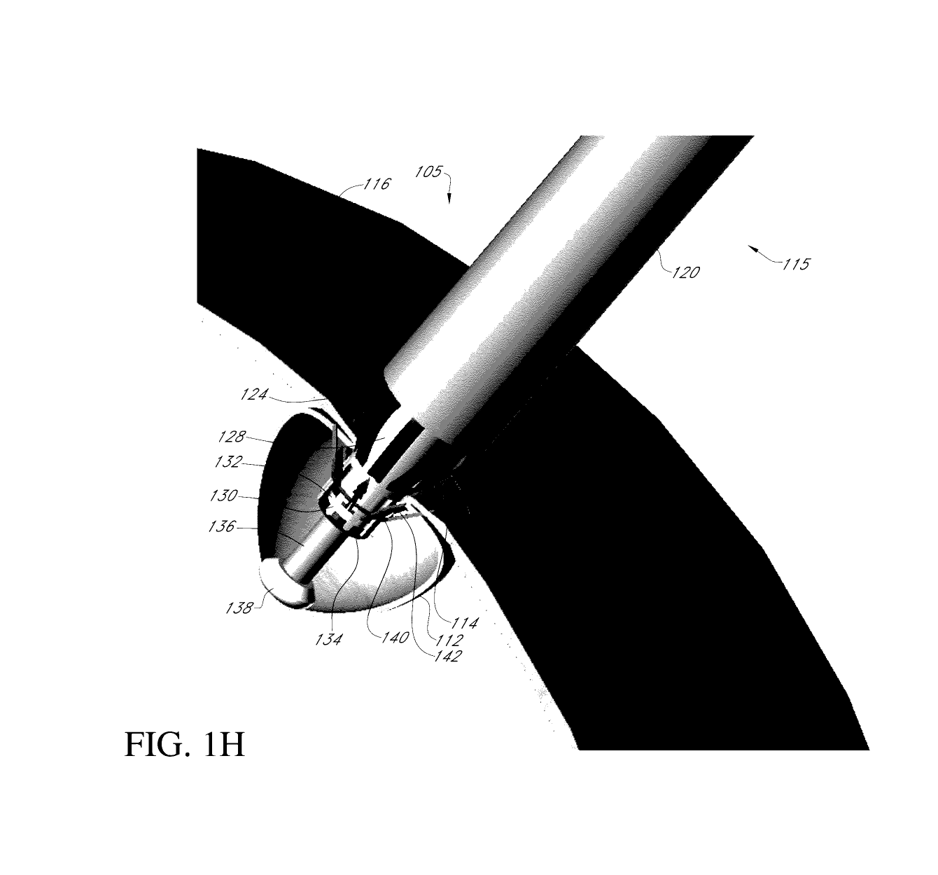

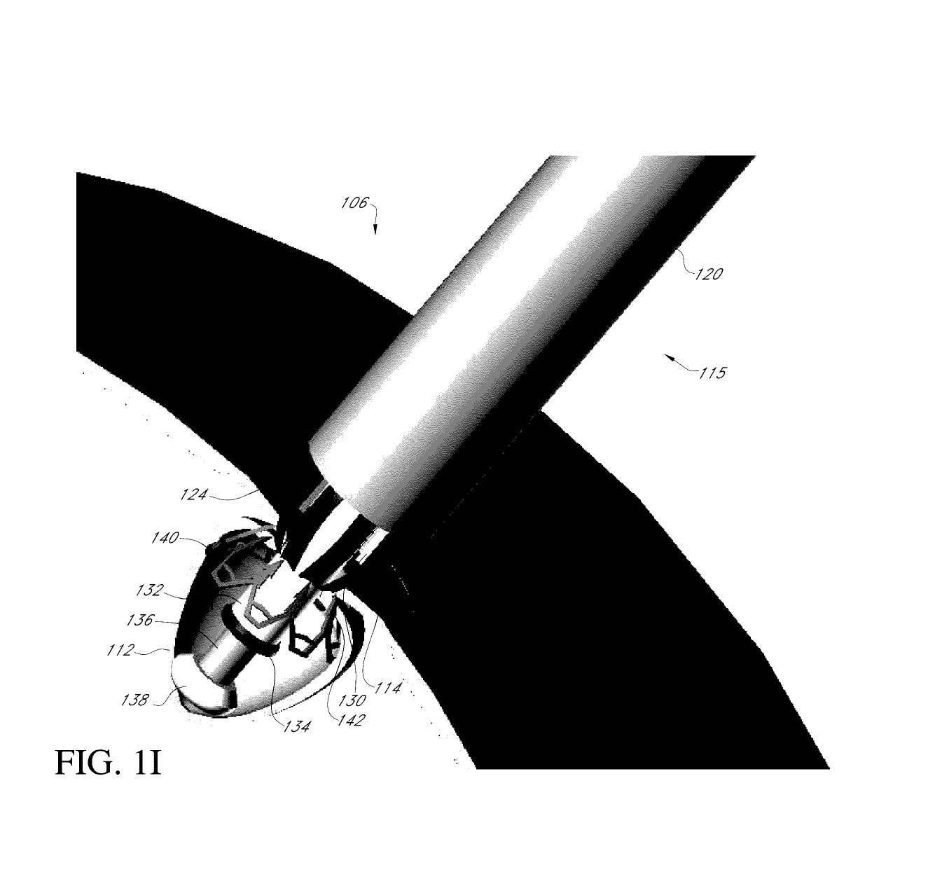

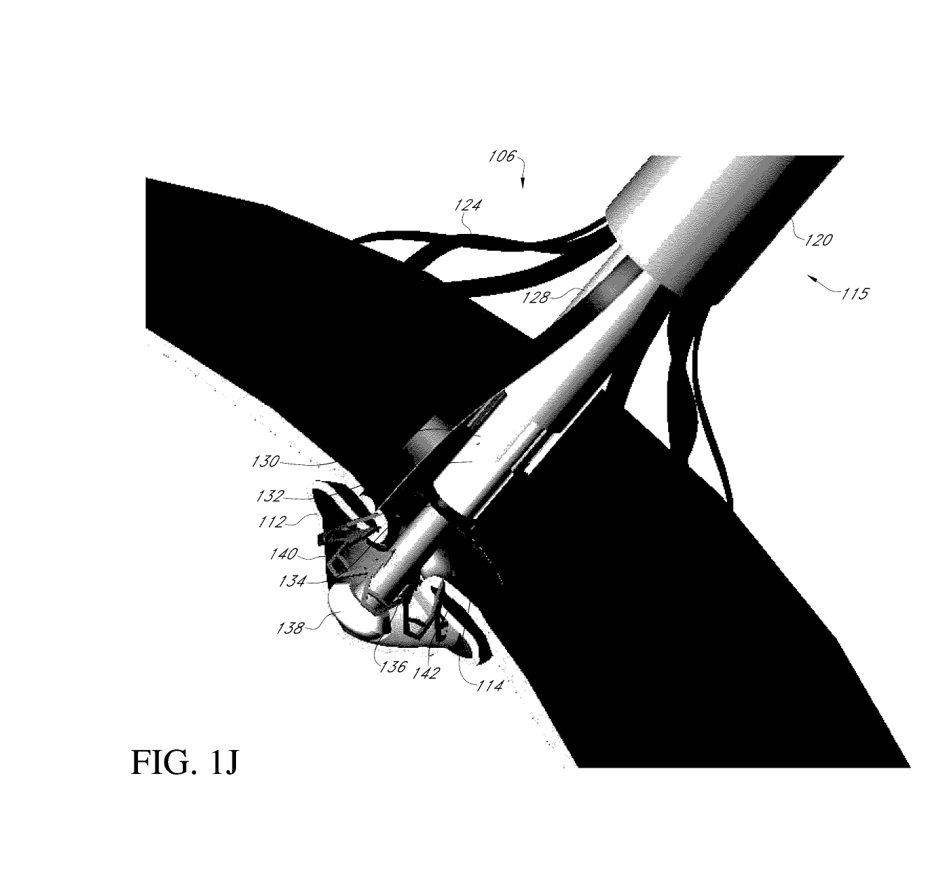

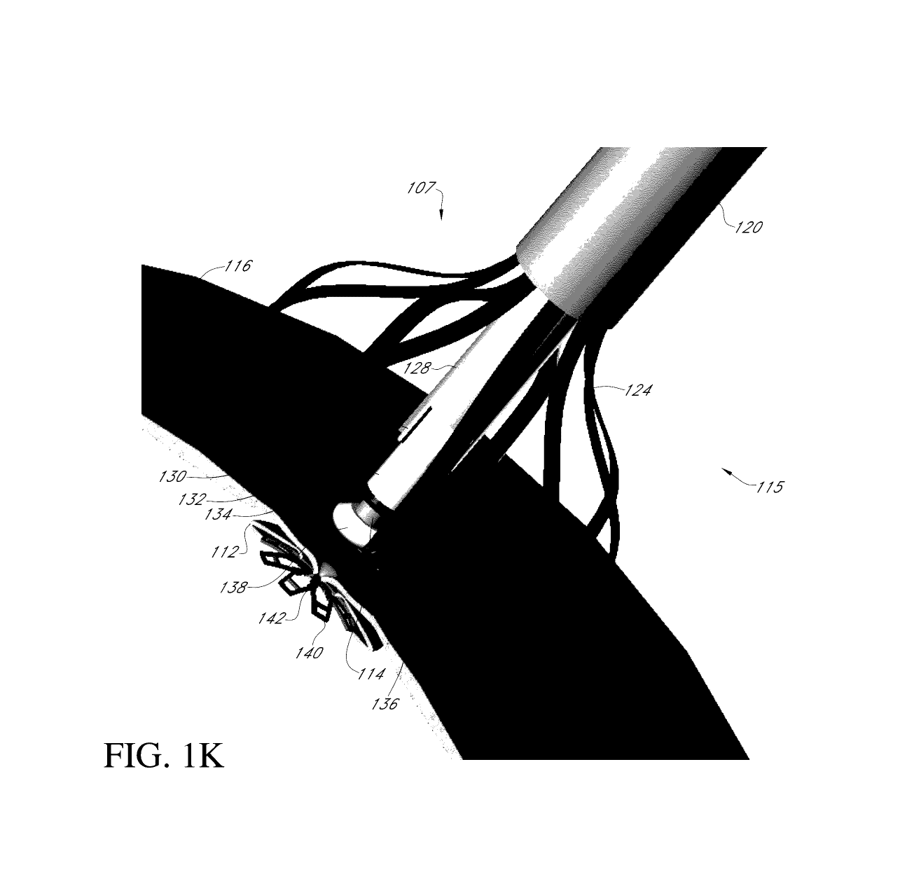

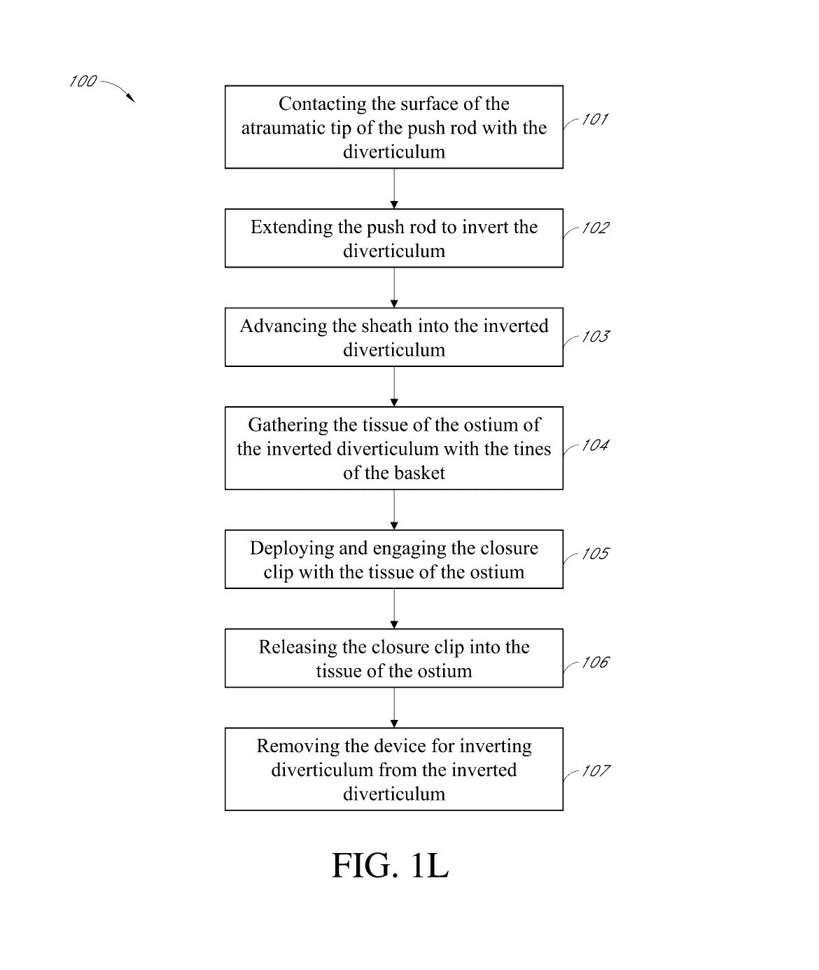

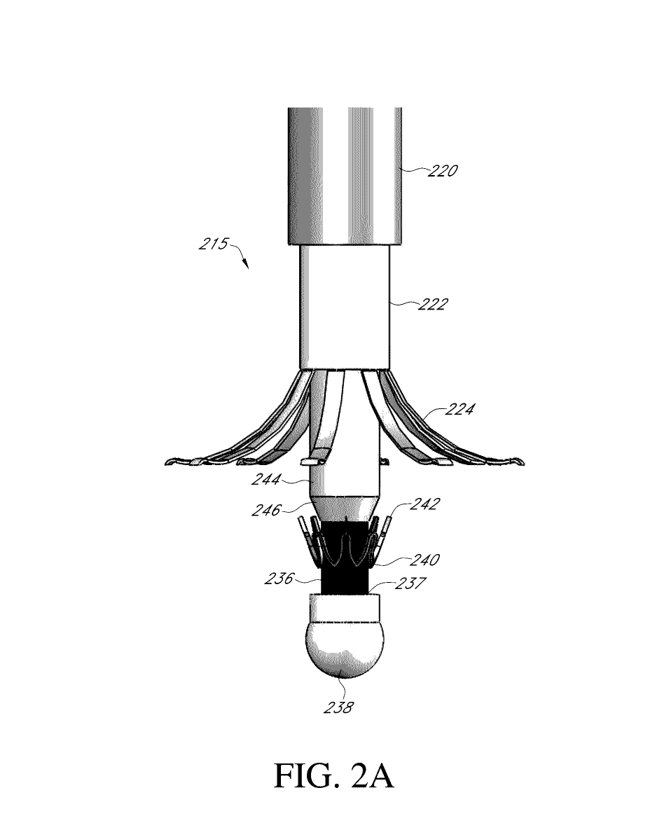

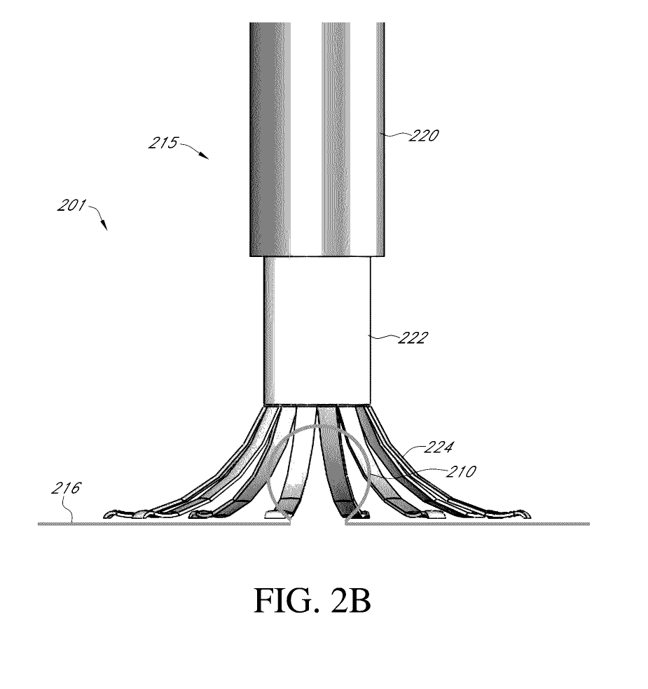

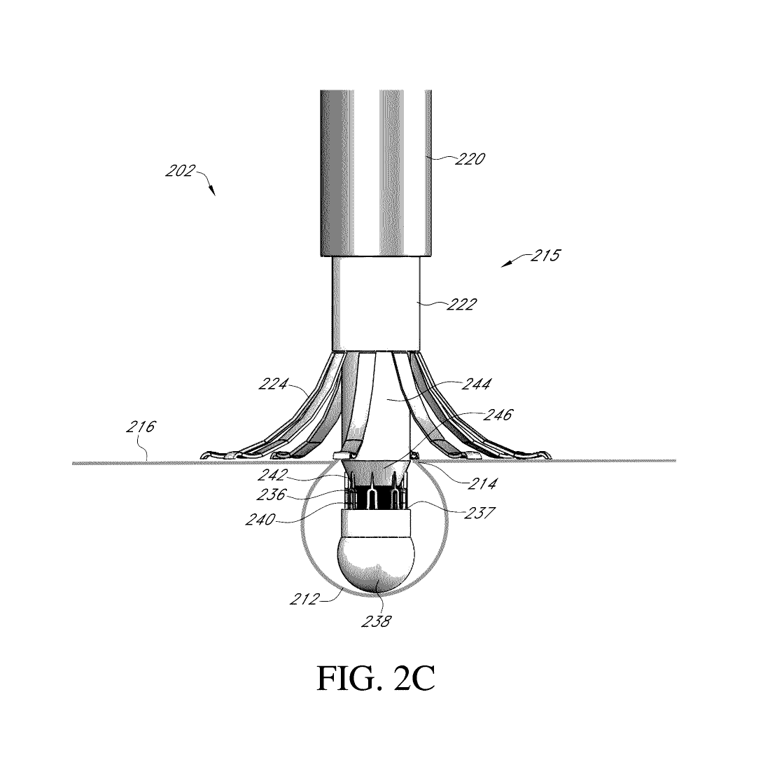

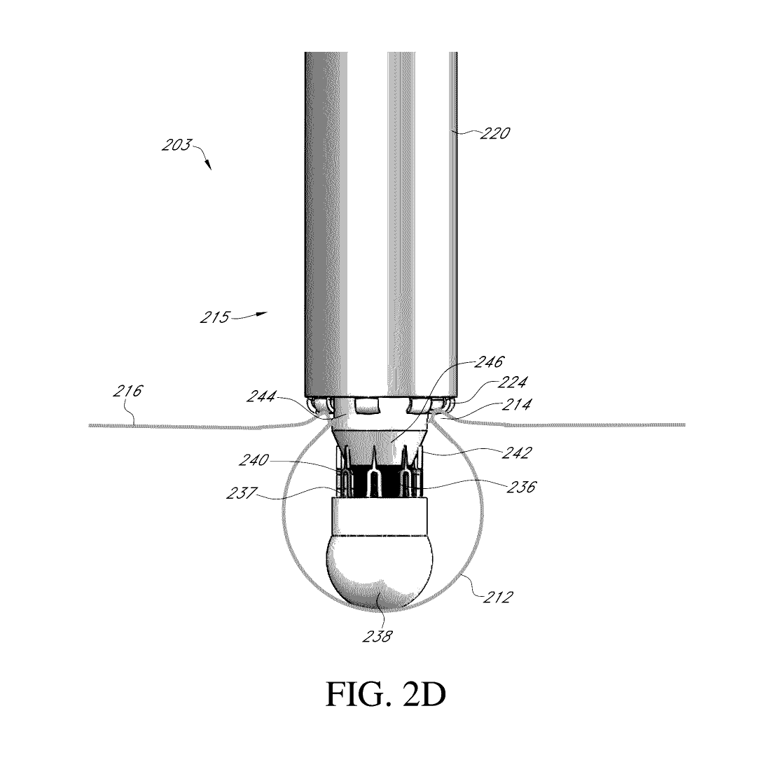

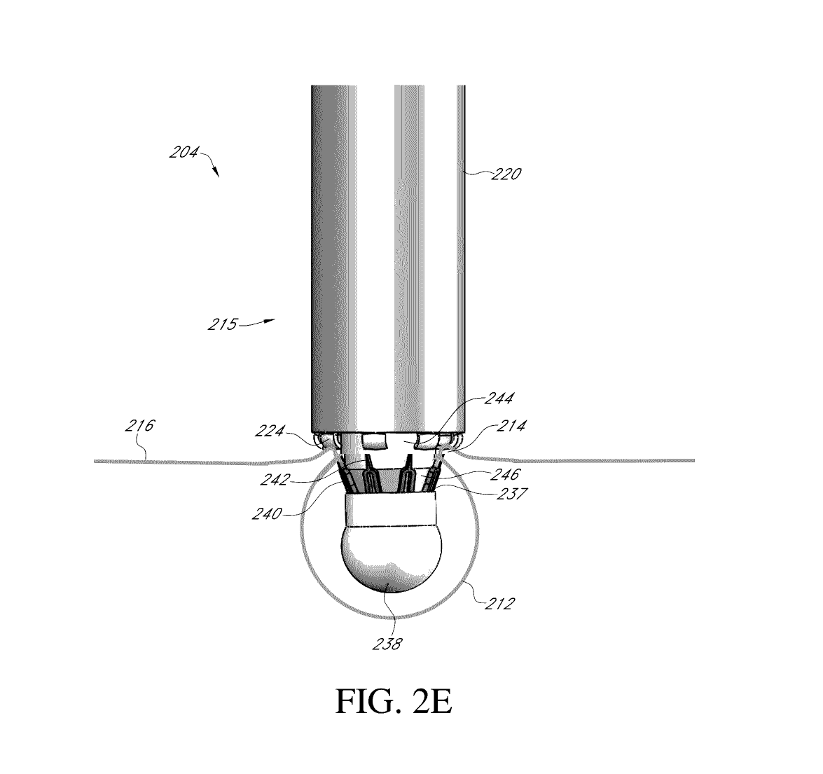

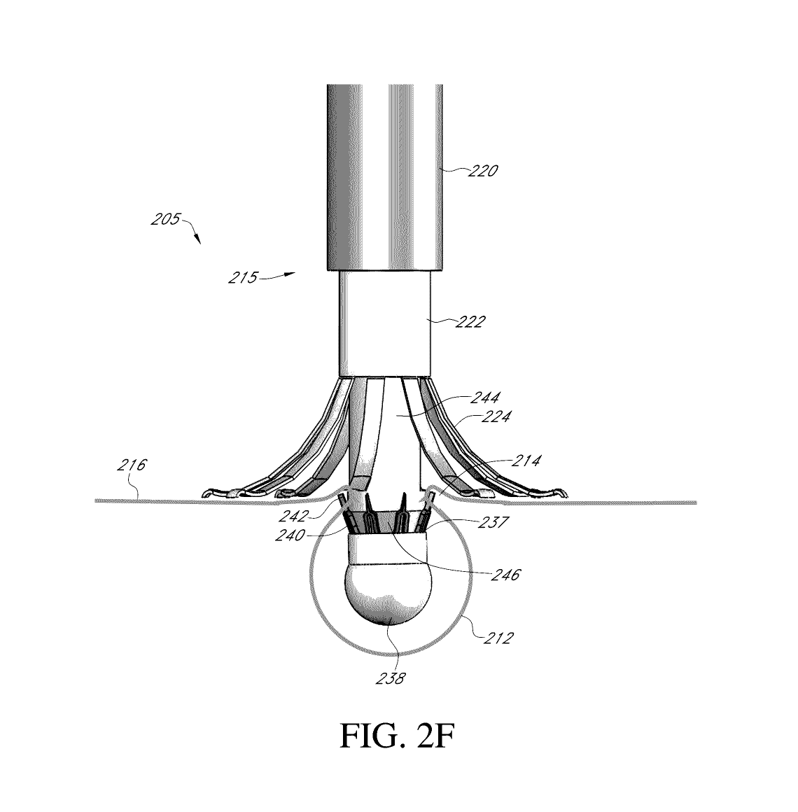

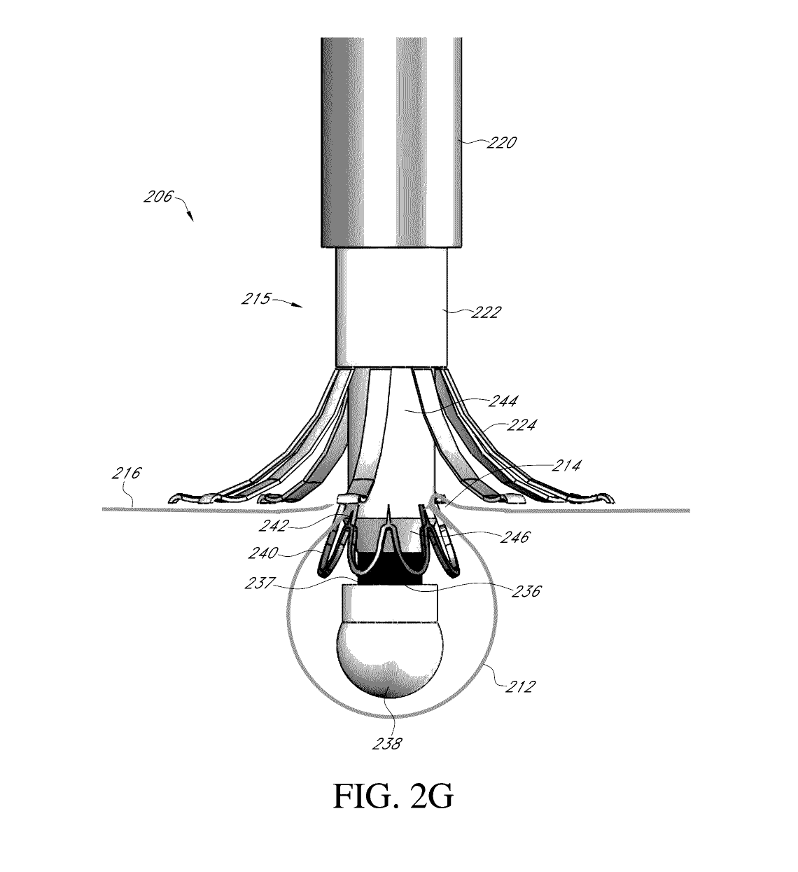

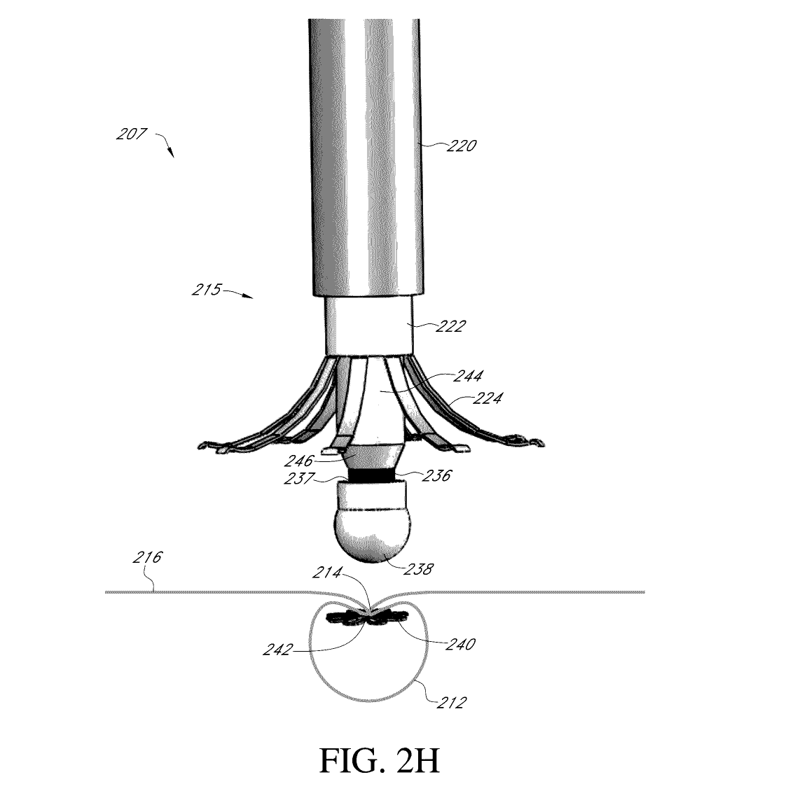

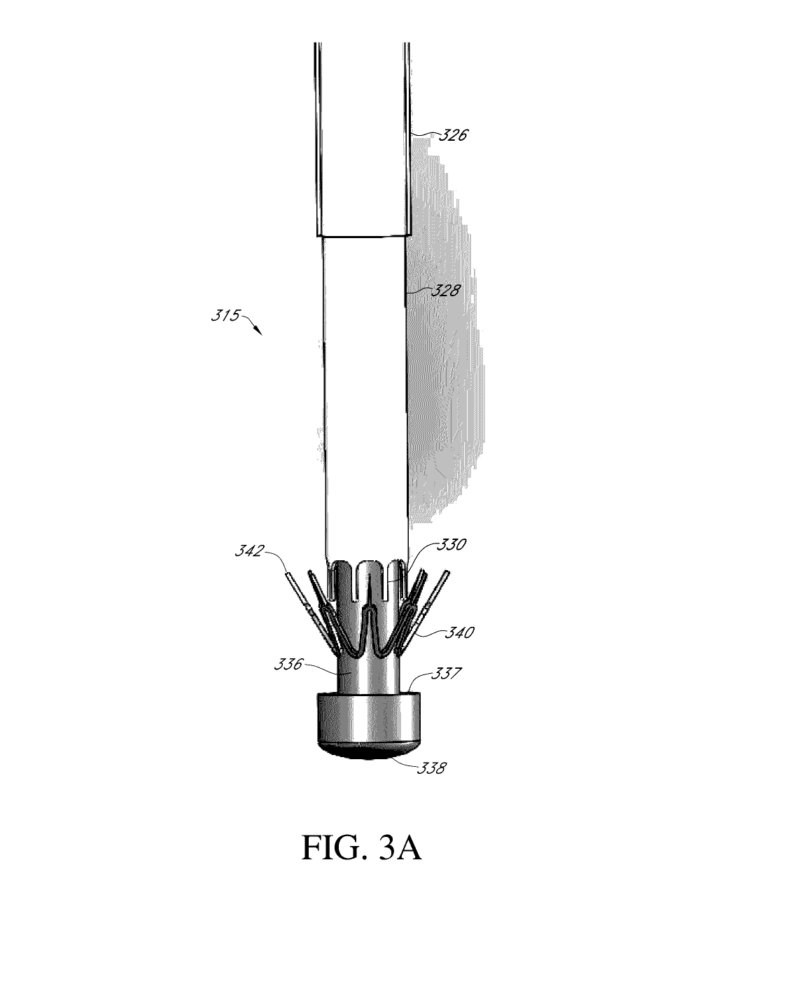

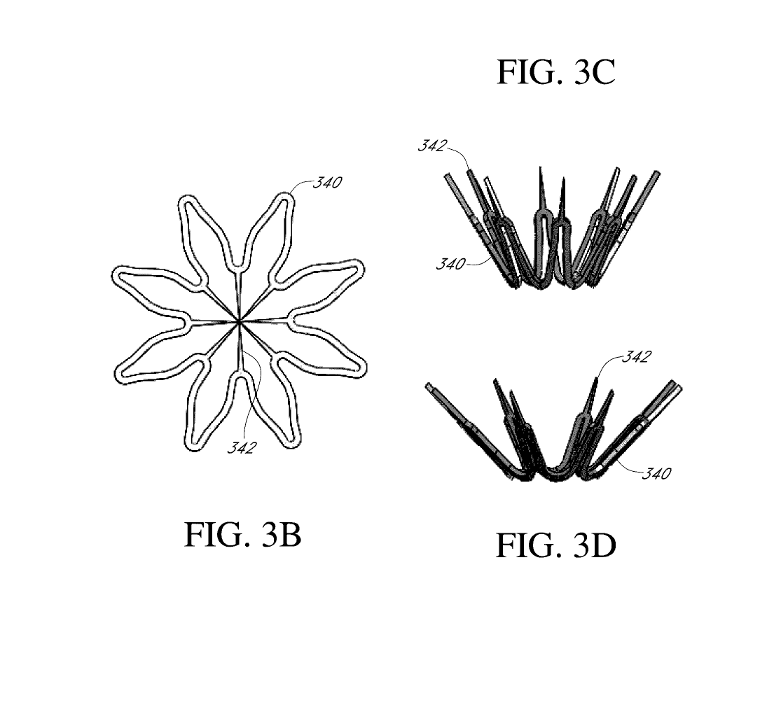

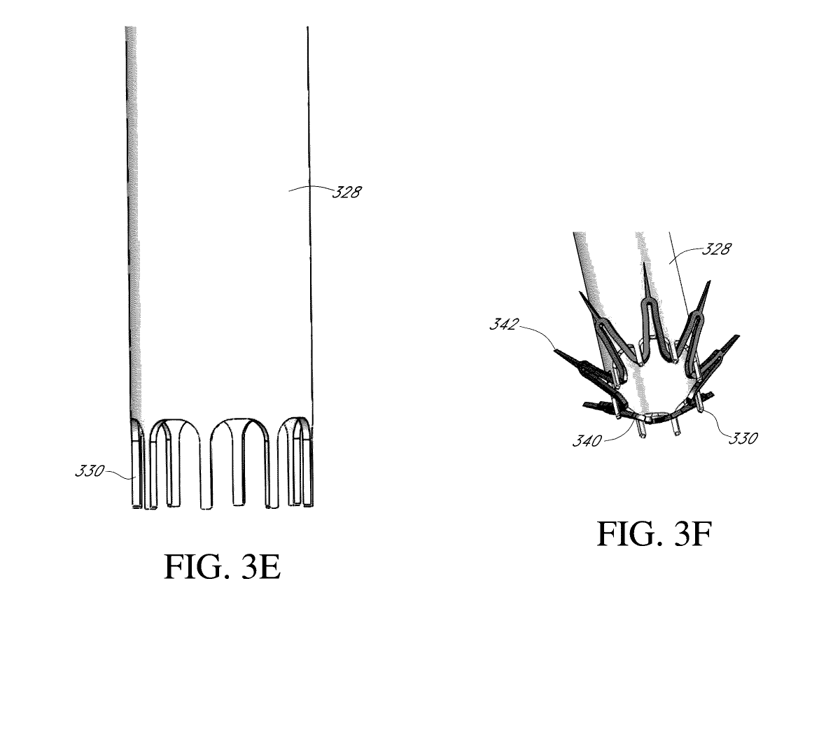

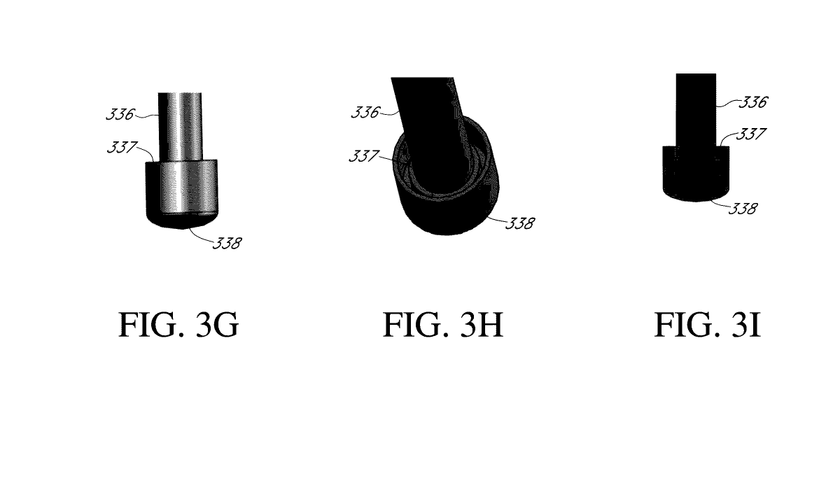

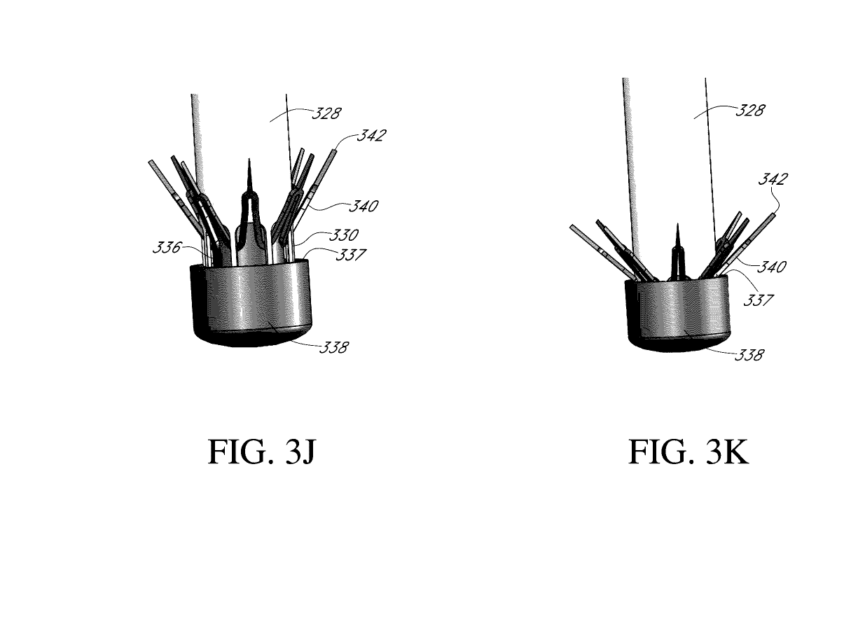

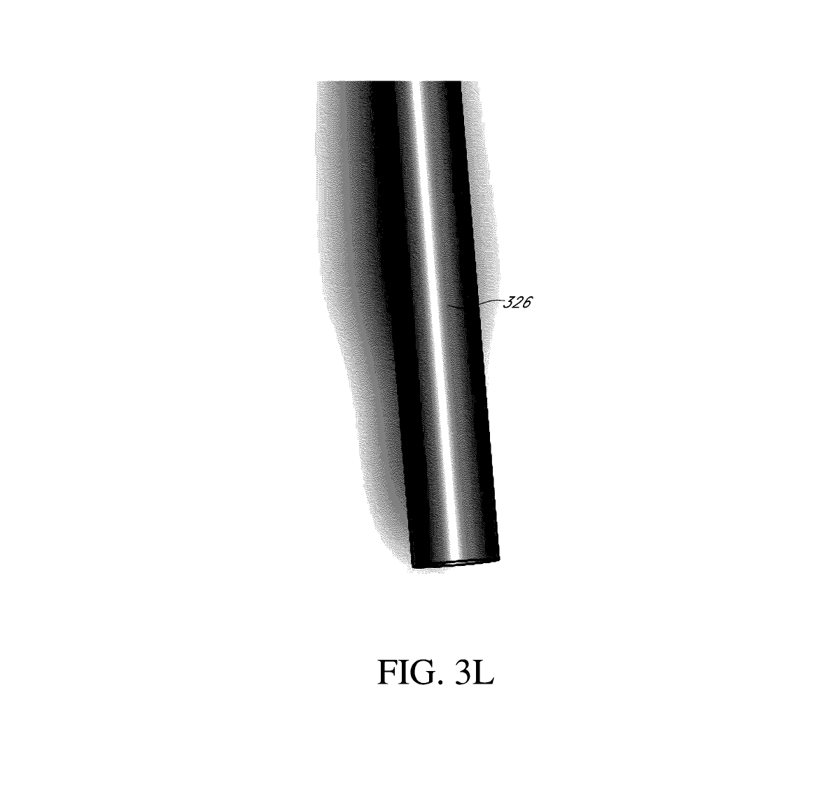

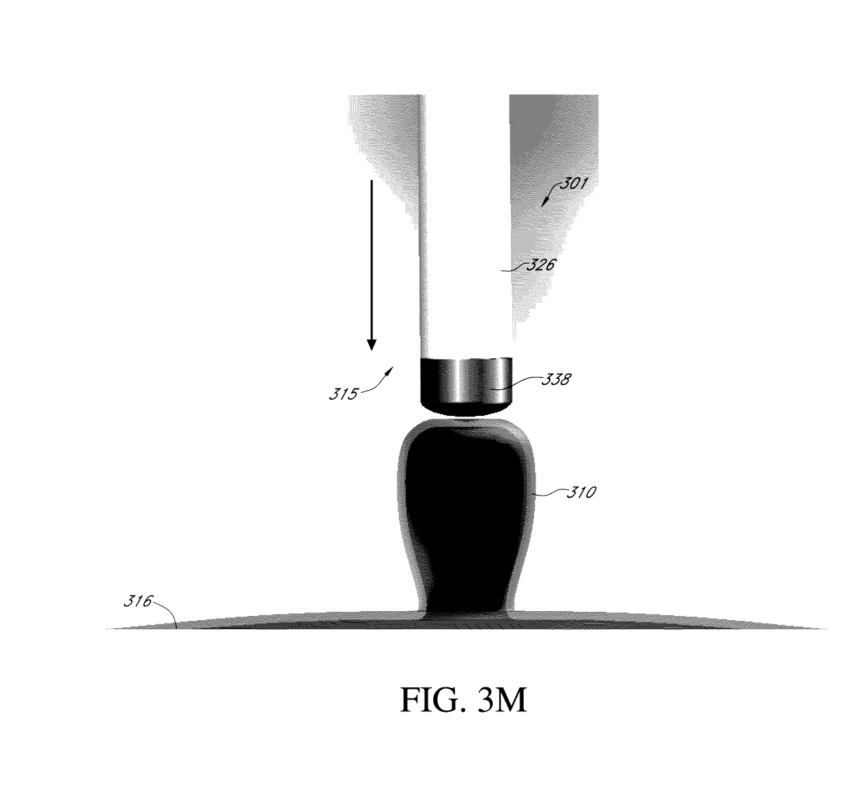

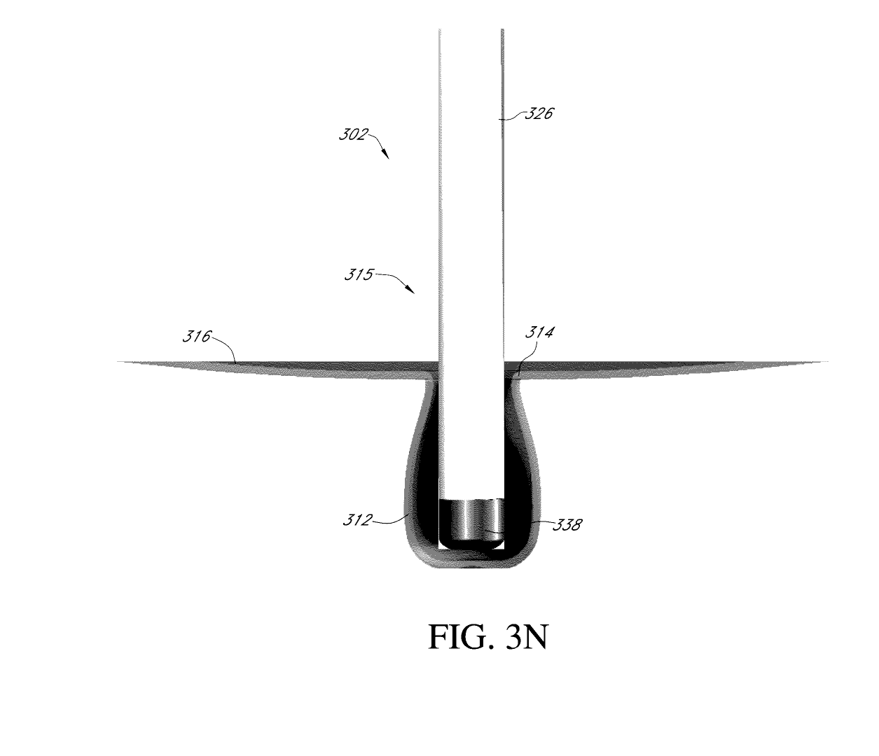

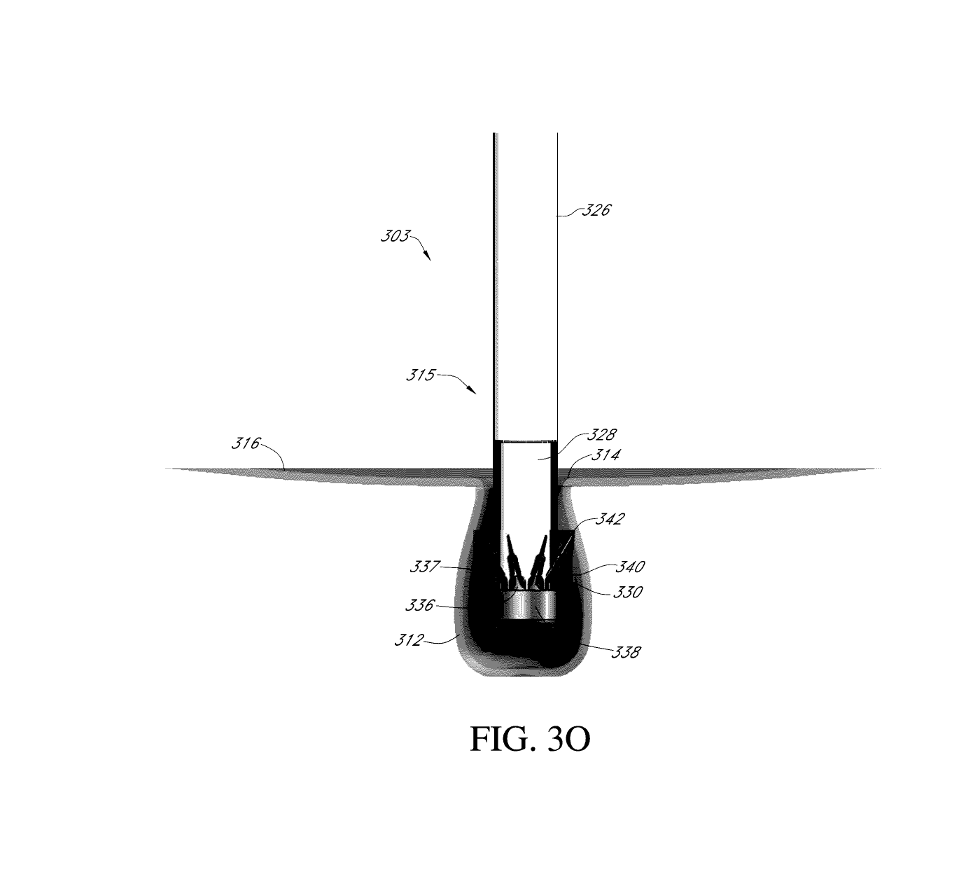

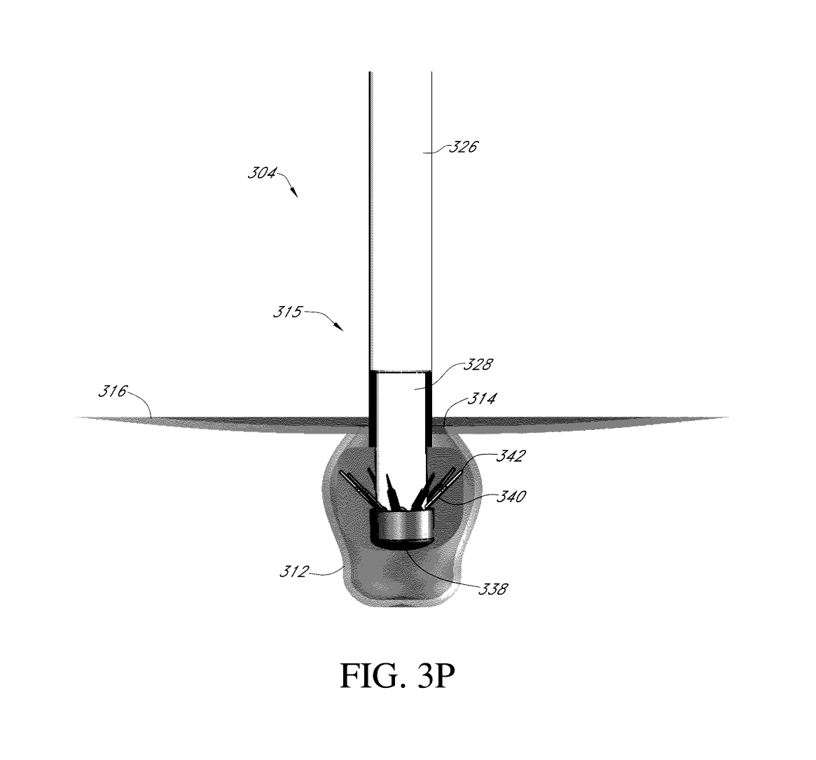

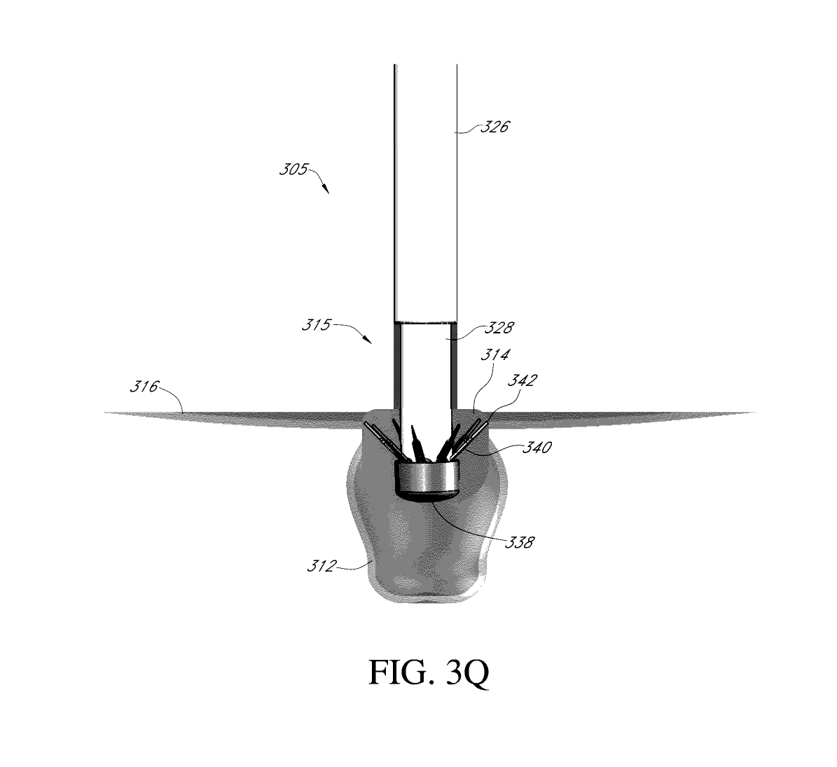

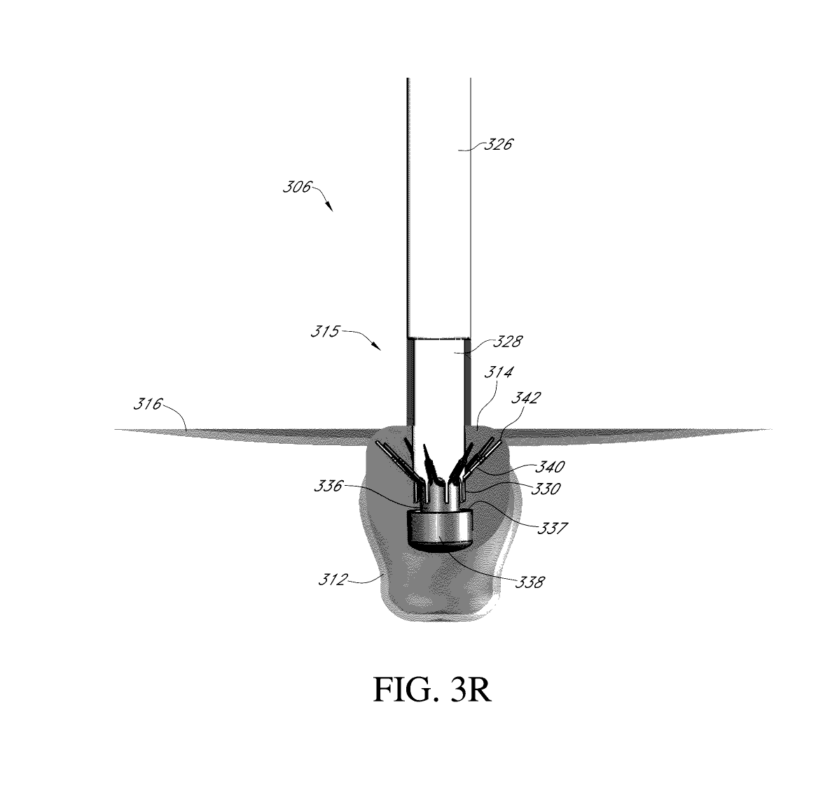

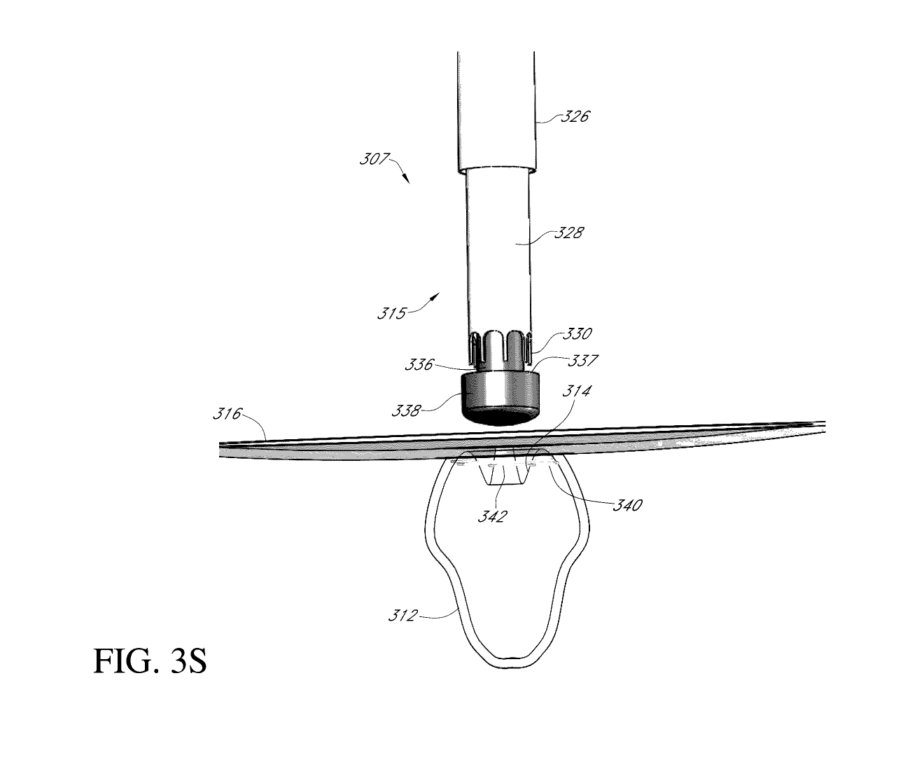

FIGS. 1A-G, 2A-H, and 3A-R illustrate a plurality of views of a device for inverting diverticulum in a method for treating diverticulum disease. FIGS. 1A-G illustrate a plurality of views of embodiments of a device for inverting diverticulum 115 in an embodiment of a method for inverting diverticula 100. FIGS. 2A-H illustrate a plurality of views of another embodiment of a device for inverting diverticulum 215 in some embodiments of a method of inverting diverticula. Finally, FIGS. 3A-R illustrate a plurality of view of another embodiment of a device for inverting diverticulum 315 in some embodiments of a method of inverting diverticula.

Turning first to the embodiment of a device for inverting diverticulum 115 illustrated in FIG. 1A, in some embodiments the device for inverting diverticulum 115 is composed of a plurality of components that are disposed coaxially about each other. As will be illustrated in the method of inverting diverticula 100, each of the components of the device for inverting diverticulum 115 are configured to be moveable relative to each other.

In some embodiments the device for inverting diverticulum 115 can include a closer 120, a basket shaft 122, a sheath 126, a clip tube 128, a tube stop 132, and a push rod 136. In some embodiments, the basket shaft 122 can further include a basket 124 at the distal end. As well, in some examples, the clip tube 128 can include a section including locking structures 130 at the distal end. As will be discussed, the locking structures 130 of the clip tube 128 can help to retain a closure clip 140. In some embodiments, the push rod 136 can further include an atraumatic tip 138 at the distal end. Each of the tubular components of the device for inverting diverticulum 115 can be composed of hardened steel.

As seen in FIG. 1A, the push rod 136 is located at the center of the device for inverting diverticulum 115. In some embodiments, the push rod 136 includes an atraumatic tip 138 at the distal end. As will be discussed below, the atraumatic tip 138 of the push rod 136 can serve to invert a target diverticulum during the method of inverting diverticula 100.

In some examples, a tube stop 132 is disposed coaxially about the push rod 136. In some variants, a flange 134 is located at the distal end of the tube stop 132 such that the flange 134 forms a wider diameter than the distal opening of the tube stop 132. The push rod 136 and the tube stop 132 can move relative to each other such that the push rod 136 can be withdrawn into the tube stop 132. In some examples, the atraumatic tip 138 has a sufficiently wide diameter such that it prevents the push rod 136 from being withdrawn entirely into the tube stop 132. As will be discussed in more detail below, the tube stop 132—in particular the flange 134 of the tube stop 132—can help to adjust the angle on which the closure clip 140 is retained on the clip tube 128 as well as adjust the angle that the closure clip 140 penetrates into the target diverticulum.

In some variants, a clip tube 128 is disposed about the tube stop 132. As noted above, in some examples, the clip tube 128 includes a plurality of locking structures 130 that are located at the distal end of the clip tube 128. The locking structures 130 can be configured to retain a closure clip 140 on the distal end of the device for inverting diverticulum 115. The clip tube 128 is configured such that it is moveable relative to the tube stop 132. In some examples, the locking structures 130 of the clip tube 128 can extend past the flange 134 of the tube stop 132. As will be discussed below, the locking structures 130 can help to retain the closure clip 140 on the device for inverting diverticulum 115. In some variants, along with the flange 134, the locking structures 130 can adjust the angle of the closure clip 140 on the distal end of the device for inverting diverticulum 115 to better allow the closure clip 140 to engage with the tissue of the target inverted diverticulum. As well, the interaction of the flange 134 and the locking structures 130 can also allow the closure clip 140 to be deployed into the inverted diverticulum.

In some embodiments, a sheath 126 can be disposed about the clip tube 128. In some examples, the diameter of the sheath 126 should be wide enough such that it can accommodate the clip tube 128 and the attached closure clip 140. In some examples, the purpose of the sheath 126 is to prevent the closure clip 140 or the locking structures 130 of the clip tube 128 from catching onto anything prior to deployment or placed into the inverted diverticulum.

In some examples, the device for inverting diverticulum 115 can further include a basket shaft 122. As illustrated in FIG. 1A, in some variants, the basket shaft 122 can include a basket 124 located at the distal end of the basket shaft 122. As the basket shaft 122 is configured to be moveable relative to the sheath 126, the basket shaft 122 can be extended or withdrawn proximal and/or distal to the other components of the device for inverting diverticulum 115. As will be described in more detail below, the basket 124 is configured to retain the tissue about the ostium of the inverted diverticulum. This can help to better deploy the closure clip 140 into the target tissue.

Lastly, in some embodiments, the device for inverting diverticulum 115 can further include a closer 120. In some examples, the closer 120 is configured to be disposed about the internal components of the device for inverting diverticulum 115. As the closer 120 is moveable relative to the basket shaft 122, the closer 120 can extend distally to cover and retain the basket 124 of the basket shaft 122. In some examples this can help to maintain the minimal profile of the device for inverting diverticulum 115 prior to use of the device in the method of inverting diverticula 100.

As noted above, the various components of the device for inverting diverticulum 115 can be moveable relative to each other. As well, in order to maintain a minimal profile of the device for inverting diverticulum 115 prior to use of the device for inverting diverticulum 115 in the method of inverting diverticula 100, the closer 120 can be used to retain the basket 124 of the basket shaft 122. Similarly, to prevent the inadvertent deployment or interaction of the closure clip 140 with the surrounding environment, the sheath 126 can be disposed about the clip tube 128, tube stop 132, and the closure clip 140 retained in between. FIG. 1B illustrates the initial configuration of the device for inverting diverticulum 115 after the basket 124 is released from the closer 120. As described, the sheath 126 is disposed about the clip tube 128, tube stop 132, and the closure clip 140.

Once the device for inverting diverticulum 115 has been inserted into the body, the device can be used to treat a diverticulum. FIG. 1L illustrates a flow chart of the method of inverting diverticula 100. Each of the steps of the method of inverting diverticula 100 are illustrated in FIGS. 1C-K. In the method of inverting diverticula 100, the device for inverting diverticulum 115 is used to treat a diverticulum 110 that is located on the surface of the outer wall of colon 116

FIG. 1C illustrates step 101 in the method of inverting diverticula 100. As illustrated, the outer wall of colon 116 includes a diverticulum 110 that protrudes from the diverticulum 110. In some embodiments, in step 101, the atraumatic tip 138 of the push rod 136 is extended to contact the top surface of the diverticulum 110. In some examples, the atraumatic tip 138 enters substantially perpendicular to the colon surface. Initial contact adjustments may be necessary in order to evenly contact the diverticulum 110.

Next, as illustrated in FIG. 1D, the diverticulum 110 is inverted at step 102. In some embodiments, as can be seen, the push rod 136 can extend distally such that the atraumatic tip 138 compresses the diverticulum 110 to force it to invert. As the diverticulum 110 inverts, the outer lip of the tissue of the diverticulum 110 can form an ostium that can provide the closure clip 140 with tissue to engage with.

Once the diverticulum 110 has been inverted, as illustrated in FIG. 1E, step 103 involves fully forming the inverted diverticulum 112 and preparing the basket 124 to engage with the surrounding tissue of the ostium 114. As can be seen, after the diverticulum 110 is inverted with the atraumatic tip 138 of the push rod 136, the sheath 126 with the retained clip tube 128, tube stop 132, and closure clip 140 can be further advanced into the inverted diverticulum 112. The advancing of the device into the inverted diverticulum 112 better allows the tines of the basket 124 to engage with the ostium tissue 114 in tension (not pictured in FIG. 1E). In some examples, prior to the basket 124 touching the tissue of the ostium 114, the diameter of the basket 124 can be adjusted to 2-3 mm greater than the ostium 114. In some embodiments, once the basket 124 engages with the ostium 114 tissue, the outer wall of colon 116 is depressed by approximately 1 inch.

Next, FIG. 1F illustrates step 104 which describes tissue acquisition by the basket 124 once the device for inverting diverticulum 115 is in position. As illustrated, while the tissue of the inverted diverticulum 112 and ostium 114 are still under tension by the inserted distal end of the device for inverting diverticulum 115, the basket 124 is closed about the ostium 114 to draw up the tissue evenly. In some embodiments, this is accomplished by advancing the closer 120 distally past the tines of the basket 124. As the closer 120 is advanced, the tines of the basket 124 are drawn together to capture the tissue of the ostium 114. In some embodiments, if the tines of the basket 124 slip or the creases of the captured tissue are largely asymmetrical, the basket 124 can be opened and step 104 can be repeated.

Once the tissue of the ostium 114 has been captured by the basket 124, step 105 as illustrated in FIGS. 1G-H disclose deploying and engaging of the closure clip 140 with the tissue of the ostium 114 while located in the inverted diverticulum 112. As illustrated in FIG. 1G, the inverted diverticulum 112 can be expanded (e.g. with gas) to allow the closure clip 140 to expand. As described above, the attached closure clip 140 can be expanded to allow the clip tines 142 to flare outwards by withdrawing the sheath 126 in a proximal direction. In some embodiments, the withdrawn sheath 126 allows the clip tines 142 of the closure clip 140 to flare outwards. In some embodiments, as illustrated in FIG. 1H, the clip tube 128 and the tube stop 132 are withdrawn proximally to engage with the tissue of the inverted diverticulum 112 and ostium 114. As discussed, in some examples, because the inverted diverticulum 112 is expanded with gas, this prevents the inverted diverticulum 112 tissue from bunching up over the tines. As illustrated, the clip tines 142 of the closure clip 140 are targeted to penetrate into the outer wall of colon 116.

In some examples, once the closure clip 140 has penetrated into the tissue, the method of inverting diverticula 100 can further include step 106 which illustrates the releasing of the closure clip 140 into the tissue. As illustrated in FIG. 1I, the inverted diverticulum 112 is longer inflated (e.g. the gas can be stopped). To release the closure clip 140 into the target tissue, the clip tube 128 can first be withdrawn proximally. In some examples, the proximal withdrawing of the locking structures 130 releases the closure clip 140 from the angle on the clip tube 128 and allows the closure clip 140 to begin to rotate into its final position. The tube stop 132 is not withdrawn so as to maintain the closure clip 140 on the device. Next, as illustrated in FIG. 1J, after the clip has rotated into its final position (e.g. planar position), the tube stop 132 is withdrawn in a proximal direction. In some examples, the tube stop 132 and the flange 134 slips through the center of the inserted closure clip 140. As well, as illustrated in FIG. 1J, the basket 124 can be opened to release the tissue of the ostium 114. In some embodiments (not pictured), the closure clip 140 can be sprung to the clip tube 128 such that the withdrawal of the tube stop 132 and flange 134 can be self-driven.

Finally, the device for inverting diverticulum 115 can be removed from the inverted diverticulum 112 in step 107 as illustrated in FIG. 1K. To remove the device for inverting diverticulum 115 from the inverted diverticulum 112, the push rod 136 and atraumatic tip 138 can be withdrawn gently to pull it past the implanted closure clip 140. As seen in FIG. 1K, once the push rod 136 and atraumatic tip 138 are removed from the inverted diverticulum 112, the closure clip 140 is allowed to rotate such that it is fully flattened. In some examples, as the closure clip 140 flattens, the clip tines 142 of the closure clip 140 capture the tissue of the ostium 114 so as to flatten and secure the inverted diverticulum 112 closed on the outer wall of colon 116.

FIGS. 2A-H illustrates another embodiment of the device for inverting diverticulum 215. FIG. 2A illustrates a side perspective of the distal end of the device for inverting diverticulum 215.

Turning now to another embodiment of a device for inverting diverticulum 215, in some embodiments the device for inverting diverticulum 215 is composed of a plurality of components that are disposed coaxially about each other. Similar to the method of inverting diverticula 100, in the method of inverting diverticula 200 as illustrated in FIGS. 2B-H, each of the components of the device for inverting diverticulum 215 are configured to be moveable relative to each other.

In some embodiments, the device for inverting diverticulum 215 can include a closer 220, a basket shaft 222, a ramp tube 244, and a push rod 236. As can be seen, the device for inverting diverticulum 215 is largely similar to the device for inverting diverticulum 115 with a few adjustments. For example, in some embodiments, the device for inverting diverticulum 215 includes a ramp tube 244 that can further include a ramped portion 246 at the distal end of ramp tube 244. Unlike the closure clip 140 in the device for inverting diverticulum 115 that is attached to the locking structures 130 of the clip tube 128, the closure clip 240 is disposed about the push rod 236. In some embodiments, the atraumatic tip 238 of the device for inverting diverticulum 215 can further include an inner opening 237. Each of the tubular components of the device for inverting diverticulum 115 can be composed of hardened steel.

As illustrated in FIG. 2A, the push rod 236 is located at the center of the device for inverting diverticulum 215. In some embodiments, the push rod 236 includes an atraumatic tip 238 at the distal end. In some examples, at the connection point between the push rod 236 and the atraumatic tip 238, the outer surface of the push rod 236 and the inner surface of the atraumatic tip 238 can form an inner opening 237. In some examples, the inner opening 237 can be configured to accommodate a portion of the closure clip 240 to adjust the angle that the closure clip 240 is located on the push rod 236. In other embodiments, the inner opening 237 of the atraumatic tip 238 can help to keep the closure clip 240 in a “safe” position prior to delivery. As will be discussed below, when it is ready to deliver the closure clip 240, the atraumatic tip 238 can be withdrawn to guide the closure clip 240 into the ramped position.

In some examples, the ramp tube 244 is disposed coaxially about the push rod 236. In some variants, the ramp tube 244 includes a ramped portion 246 that is located at the distal end of the ramped portion 246. The ramp tube 244 can be moveable relative to the push rod 236. In some examples, the ramped portion 246 of the ramp tube 244 can move an attached closure clip 240 into the inner opening 237 of the atraumatic tip 238 to alter the angle of the closure clip 240. The ramped portion 246 can help to adjust the angle on which the closure clip 240 is retained on the push rod 236 as well as to adjust the angle that the closure clip 240 penetrates into the target diverticulum. The ramped portion 246 can flare the clip into delivery position.

In some variants, the basket shaft 222 can be disposed about the ramp tube 244. In some examples, the basket shaft 222 can include a basket 224 located at the distal end of the basket shaft 222. As the basket shaft 222 is configured to be moveable relative to the ramp tube 244, the basket shaft 222 can be extended or withdrawn proximal and/or distal to the other components of the device for inverting the diverticulum 215. As will be described in more detail below, the basket 224 can be configured to retain the tissue about the ostium of the inverted diverticulum. This can help to better deploy the closure clip 240 into the target tissue.

In some embodiments, the device for inverting diverticulum 215 can further include a closer 220. In some examples, the closer 220 is configured to be disposed about the internal components of the device for inverting diverticulum 215. As the closer 220 is moveable relative to the basket shaft 222, the closer 220 can extend distally to cover and retain the basket 224 of the basket shaft 222. In some examples, this can help to maintain the minimal profile of the device for inverting diverticulum 215 prior to use of the device in the method of inverting diverticula.

The device for inverting diverticulum 215 can be used to treat a diverticulum. FIGS. 2B-H illustrate one embodiment of the method of inverting diverticula. The method of inverting diverticula is generally similar to the method of inverting diverticula 100.

FIG. 2B illustrates step 201 in the method of inverting diverticula. As illustrated, in some embodiments, after the device for inverting diverticulum 215 is inserted through the trocar, the closer 220 can be withdrawn in a proximal direction to release the 22 and the basket 224. The device for inverting diverticulum 215 can then be advanced until the basket 224 is centered on the target diverticulum 210.

Next, at FIG. 2C, the method of inverting diverticula can include step 202 wherein the atraumatic tip 238 on the push rod 236 can be advanced in a distal direction to invert the diverticulum 210. In some embodiments, the distal end of the atraumatic tip 238 can be spring loaded to prevent excessive force from being placed on the diverticulum 210 and in order to accommodate diverticulum 210 of different sizes. In some examples, at step 202, the closure clip 240 is retracted into the inner opening 237 of the atraumatic tip 238. In this configuration, the clip tines 242 is in a “safe” position, such that the clip tines 242 do not interfere with any tissue as it is inserted into the inverted diverticulum 112. In some examples, the inverted diverticulum 212 can be inflated (e.g. with gas) to allow better presentation of the ostium 214 to the closure clip 240.

In some embodiments, the method of inverting diverticula can include step 203 illustrated in FIG. 2D. Here, the basket 224 of the basket shaft 222 is closed around the tissue of the ostium 214 formed from the inverted diverticulum 212 on the outer wall of colon 216. In some examples, the basket 224 can grab the tissue by advancing the closer 220 in a distal direction until it completely covers the length of the basket 224. This can allow the basket 224 to pull and secure the healthy tissue of the ostium 214 around the ramp tube 244.

As illustrated in FIG. 2E, the method of inverting diverticula can include step 204 wherein the closure clip 240 is ramped into a “deploy” position. In some examples, at step 204, the push rod 236 is withdrawn in a distal direction with respect to the ramp tube 244. The ramped portion 246 of the ramp tube 244 can guide the closure clip 240 into the “deploy” position. In some examples, in the “deploy” position, the clip tines 242 of the closure clip 240 are flared outwards and engage with the gathered tissue at the ostium 214 of the inverted diverticulum 212. In some embodiments (not illustrated) the closure clip 240 can be released by continuing to withdraw the atraumatic tip 238 in a proximal direction. While the inner ledge 237 holds the clip in place, the movement of the atraumatic tip 238 is under the clip and ejects the closure clip 240 into the tissue as the closure clip 240 is moved along the ramped portion 246 of the ramp tube 244. Once the closure clip 240 is free of the inner ledge 237, it can begin to expand outwardly to return to its original planar shape.

Once the clip tines 242 of the closure clip 240 has been inserted into the tissue of the ostium 214, the method of inverting diverticula can proceed to step 205. As illustrated in FIG. 2F, the closer 220 can be withdrawn in a distal direction. As the closer 220 is withdrawn, the basket 224 can be opened—thereby releasing the gathered tissue of the ostium 214 around the neck of the inverted diverticulum 212.

In some embodiments, the method of inverting diverticula can then include step 206 as illustrated in FIG. 2G. Once the clip tines 242 of the closure clip 240 are secured in the tissue of the ostium 214, the push rod 136 can be advanced in a distal direction. In doing so, the closure clip 240 is released from the inner opening 237 of the atraumatic tip 238. This can allow the closure clip 240 to rotate with the clip tines 242 in gripping tissue around the ramp tube 244.

Once the closure clip 240 has been released from the inner opening 237 of the atraumatic tip 238, the method of inverting diverticula can proceed to step 207 where the device for inverting diverticulum 215 is retracted from the inverted diverticulum 112. In some embodiments, once the closure clip 240 has been released, the ramp tube 244, the ramped portion 246 of the ramp tube 244, the push rod 236, and the atraumatic tip 238 of the push rod 236 can be retracted through the ostium 214 of the inverted diverticulum 112. In some examples, once the distal end of the device for inverting diverticulum 215 has been fully retracted, the closure clip 240 can closed to its natural flat shape and grip the healthy tissue of the ostium 214 together with the clip tines 242.

Finally, FIGS. 3A-S illustrate another embodiment of the device for inverting diverticulum 315. FIG. 3A illustrates a side perspective of the distal end of the device for inverting diverticulum 315. FIGS. 3B-L illustrate a plurality of views of the components in the device for inverting diverticulum 315. Many of the illustrations of the components of the device for inverting diverticulum 315 can be similarly applicable to the device for inverting diverticulum 115 and device for inverting diverticulum 215 discussed above.

The device for inverting diverticulum 315 has elements that resemble or are similar to the device for inverting diverticulum 115 and device for inverting diverticulum 215 described above. Accordingly, numerals used to identify features of the device for inverting diverticulum 115 and device for inverting diverticulum 215 are incremented by a factor of one hundred to identify like features of the device for inverting diverticulum 315. This numbering conventional generally applies to the remainder of the figures. Any component or step disclosed in any embodiment in this specification can be used in other embodiments.

In some embodiments, the device for inverting diverticulum 315 (as illustrated in FIG. 3A), can be composed of a plurality of components that are disposed coaxially about each other. As will be illustrated in the method of inverting diverticula, each of the components of the device for inverting diverticulum 315 are configured to be moveable relative to each other. Unlike the previous two embodiments, in some examples, the device for inverting diverticulum 315 does not include a basket for securing the ostium of the inverted diverticulum. As discussed above, in some embodiments, each of the tubular components of the device for inverting diverticulum 115 can be composed of hardened steel.

As seen in FIG. 3A, the push rod 336 is located at the center of the device for inverting diverticulum 315. As can be seen, in some embodiments, the closure clip 340 is disposed about the push rod 336 of the device. In some embodiments, the push rod 336 can include an atraumatic tip 338 at the distal end.

FIGS. 3B-D illustrate a plurality of views of the closure clip 340. Each of these illustrations can be applicable to any discussion of the closure clip provided above. FIG. 3B illustrates a top view of an embodiment of the closure clip 340 in its relaxed state. In its relaxed state, the closure clip 340 is flat with the clip tines 342 pointing inward. In some embodiments, this is the form the closure clip 340 will take after it has been delivered inside of the inverted diverticulum 112. In some embodiments, the closure clip 340 can have tines that are 1.88 mm. FIG. 3C illustrates the closure clip 340 as it is loaded on the locking structures 330 of the clip tube 328. In this loaded state, the closure clip 340 is in a semi-flared shape. Lastly, FIG. 3D illustrates the closure clip 340 as it is flared even more for delivery to capture the tissue in the ostium 314. As was discussed, and will be discussed below, in the various embodiments of the method of inverting diverticula, the flaring of the clip tines 342 provide the closure clip 340 with a broader reach to engage the surrounding tissue of the ostium 314.

In some examples, at the connection point between the push rod 336 and the atraumatic tip 338, the outer surface of the push rod 336 and the inner surface of the atraumatic tip 338 can form an inner opening 337. In some examples, the inner opening 337 can be configured to accommodate a portion of the closure clip 340 to adjust the angle that the closure clip 340 is located on the push rod 336. FIGS. 3G-I illustrate a side, top, and cross-sectional view of the atraumatic tip 338. As is illustrated, the inner opening 337 provided between the outer surface of the push rod 336 and the inner surface of the atraumatic tip 338 provides an opening to accommodate a portion of the closure clip 340. As well, as illustrated in FIG. 3H, in some embodiments, the inner opening 337 can include a ledge that is adjacent the outer surface of the push rod 336.

In some examples, a clip tube 328 can be disposed about the push rod 336. In some embodiments, the clip tube 328 includes locking structures 330 at the distal end of the clip tube 328. As will be discussed in more detail below, the locking structures 330 can engage with the closure clip 340 that is disposed about the push rod 336 to secure the closure clip 340 on the distal end of the device for inverting diverticulum 315. As noted above, because the clip tube 328 and the push rod 336 are moveable relative to each other, withdrawing or advancing the clip tube 328 can cause the locking structures 330 to interact with the closure clip 340 to alter the angle in which the clip tines 342 are flared on the distal end of the device.

FIGS. 3E-F illustrate the clip tube 328 and a bottom perspective view of the clip tube 328 with closure clip 340 engaged. As illustrated in FIG. 3E, the locking structures 330 can include a plurality of equally spaced structures. FIG. 3F illustrate the closure clip 340 as it is engaged with the locking structures 330. As seen, each apex of the closure clip 340 is linked around the locking structures 330 to create the loaded flare shape—wherein the clip tines 342 are flared outwards and pointed in a proximal direction.

FIGS. 3J-K provide an illustration of the interaction between the locking structures 330 of the clip tube 328, the closure clip 340, and the atraumatic tip 338 of the push rod 336. FIG. 3J illustrates the closure clip 340 as it is loaded on the clip tube 328. In some examples, as the atraumatic tip 338 is moved up towards the clip tube 328, the ledge in the inner opening 337 can bump the apices of the closure clip 340. This can push them upward to cause a larger flare. FIG. 3K illustrates the distal end of the device for inverting diverticulum 315 and the configuration of the closure clip 340 during delivery. The structure of the inner opening 337 and atraumatic tip 338 allows the closure clip 340 to be pulled into the tissue without being inverted due to the force.

Lastly, in some embodiments, the device for inverting diverticulum 315 can further include a sheath 326 that can be disposed about the clip tube 328. In some examples, as seen in FIG. 3L, the sheath 326 can be wide enough such that it can accommodate the clip tube 328 and the attached closure clip 340. In some examples, the purpose of the sheath 326 is to prevent the closure clip 340 or the locking structures 330 of the clip tube 328 from unintentionally interacting with any tissue prior to deployments of the closure clip 340. The sheath 326 therefore protects the surrounding tissue as the device for inverting diverticulum 315 is inserted. As will be seen, the sheath 326 can be removed prior to deployment.

As discussed above, the device for inverting diverticulum 315 can be used to treat a diverticulum. FIGS. 3M-S illustrate another embodiment of the method of inverting diverticula. However, as will be discussed below, unlike the method of inverting diverticula discussed above, the device for inverting diverticulum 315 in the method of inverting diverticula does not include a basket for capturing the tissue of an inverted diverticula.

FIG. 3M can illustrate step 301 in the method of inverting diverticula. As is illustrated, the outer wall of colon 316 can include a diverticulum 310 that protrudes from the surface of the colon. In some embodiments, the atraumatic tip 338 located at the distal end of the device for inverting diverticulum 315 can be used to manually invert the diverticulum 310.

FIG. 3N next illustrates step 302 which shows the distal end of the device for inverting diverticulum 315 located in the inverted diverticulum 312. The distal end of the atraumatic tip 338 can further push into the inverted diverticulum 312 such that the ostium 314 is disposed snugly about the sheath 326.

The method of inverting diverticula can then include step 303 as illustrated in FIG. 3O. In some embodiments, at step 303, the sheath 326 is withdrawn in a proximal direction such that the closure clip 340 is exposed within the inverted diverticulum 312. As discussed above, the closure clip 340 at this stage is in the configuration illustrated in FIG. 3C, wherein the closure clip 340 is in a semi-flared state. In some examples, the inverted diverticulum 312 can be inflated (e.g. with gas) to allow better presentation of the ostium 214 to the closure clip 340.

In some examples, as illustrated in FIG. 3P, the method of inverting diverticula can include step 304 wherein the push rod 336 and the atraumatic tip 338 is retracted in a distal direction. As the push rod 336 is retracted, the ledge located within the atraumatic tip 338 (not pictured) can pull the closure clip 340 in a proximal direction, causing the closure clip 340 to flare to a larger diameter. In some examples, the closure clip 340 can be in the configuration illustrated in FIG. 3D wherein the flaring of the clip tines 342 can provide the closure clip 340 with a broader reach in order to engage the surrounding tissue of the ostium 314.

Once the closure clip 340 is flared outwards, the method of inverting diverticula can then include step 305 wherein the entirety of the device for inverting diverticulum 315 is retracted in a proximal direction. In some embodiments, as illustrated in FIG. 3Q, the device for inverting diverticulum 315 can be retracted in a proximal direction, this can serve to seat the clip tines 342 of the closure clip 340 into the surrounding tissue of the ostium 314. In some examples, once the clip tines 342 are in the surrounding tissue of the ostium 314, the closure clip 340 is ready to be released.

In some examples, the method of inverting diverticula can then include step 306 in order to begin the step of releasing the closure clip 340 into the tissue of the ostium 314. As illustrated in FIG. 3R, the atraumatic tip 338 of the push rod 336 is advanced in a distal direction. This can relax the closure clip 340 from the flared configuration to a semi-flared configuration. In some examples, the advancing of the atraumatic tip 338 in the inverted diverticulum 112 can allow the closure clip 340 to be released from the locking structures 330 of the clip tube 328. In some embodiments, (not illustrated) the push rod 336 can include a ramped portion. The closure clip 340 can be released by continuing to withdraw the atraumatic tip 338 in a proximal direction to cause the closure clip 340 to be ejected from the push rod 336.

Once the closure clip 340 has been released from the locking structures 330 of the clip tube 328, the distal end of the device for inverting diverticulum 315 can be retracted from the inverted diverticulum 112. As illustrated in FIG. 3S, in some embodiments, the configuration of the closure clip 340 can allow the atraumatic tip 338 to be pulled through the center of the closure clip 340. As the distal end of the device for inverting diverticulum 315 is pulled from the ostium 314 of the inverted diverticulum 312, the closure clip 340 can close around the captured tissue of the ostium 314. In some examples, the closure clip 340 can take the form of the closure clip 340 illustrated in FIG. 3B.

As described above, in some embodiments, the above steps described for the method of inverting diverticula 100, can be used to successfully invert a diverticulum 110, 210, 310 and place a closure clip 140, 240, 340 in the inverted diverticulum 112, 212, 312. In some examples, the device for inverting diverticulum 115, 215, 315 can be configured such that the user can use a different closure clip 140, 240, 340, than initially inserted.

For example, after the healthy tissue on the ostium 114, 214, 314, has been gathered, the user can determine that the size of the closure clip 140, 240 340 required is a different size (e.g. smaller or larger) than initially presumed. This would therefore require that the clip be removed from the device and steps in the method would need to be repeated as the device with an appropriately sized clip is reinserted. Described below is an example of a device for inverting diverticulum 415 that enables the user to swap out the closure clip 440 (or even delay introduction of the closure clip 440) until the target tissue has been gathered and an assessment of the closure clip 440 requirements has been made. This same feature can allow for a significant increase in the speed in the speed of a procedure involving treating a plurality of diverticulum. In some examples, it could obviate the need for repeated removal and re-introduction of new devices containing integrated closure clips 440. In other examples, this could enable a single device for inverting diverticulum 415 to be used for treating multiple diverticulum and delivering a plurality of closure clip 440 which could produce a reduction of cost in performing the procedure.

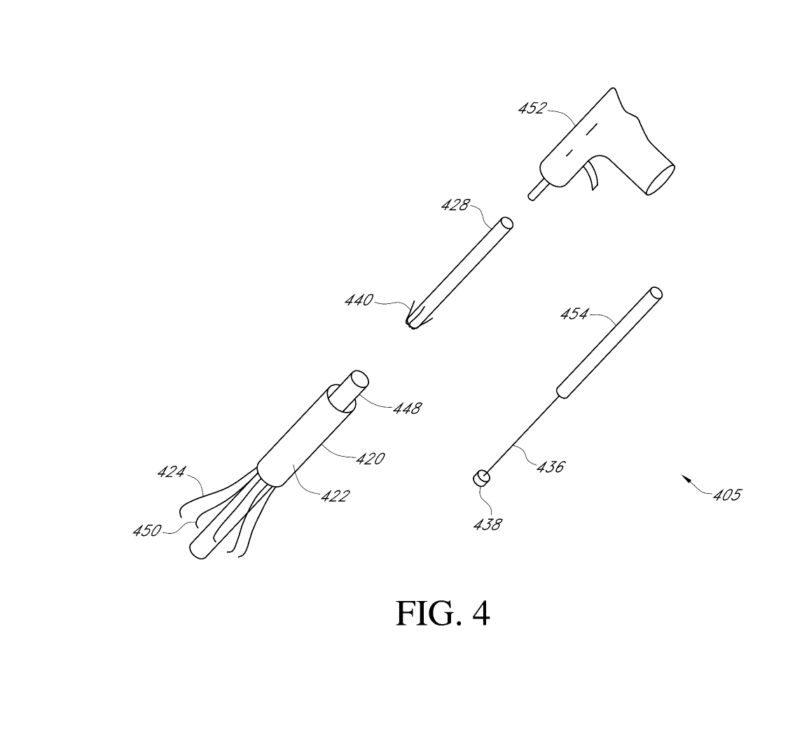

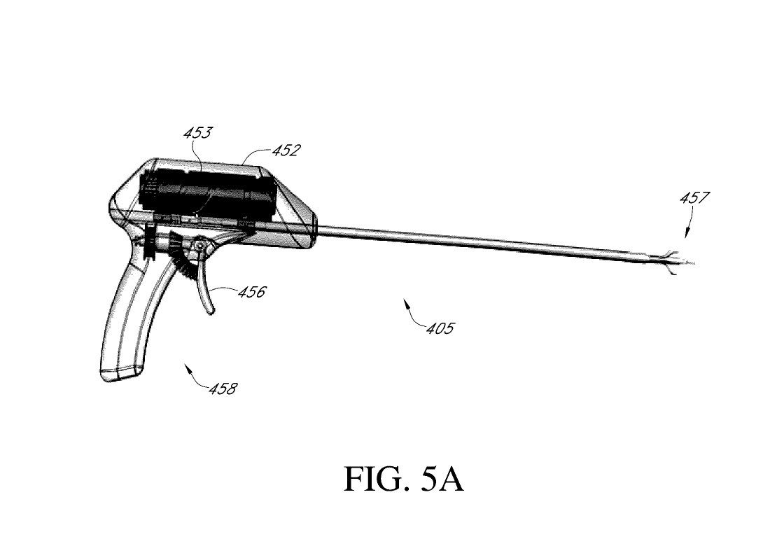

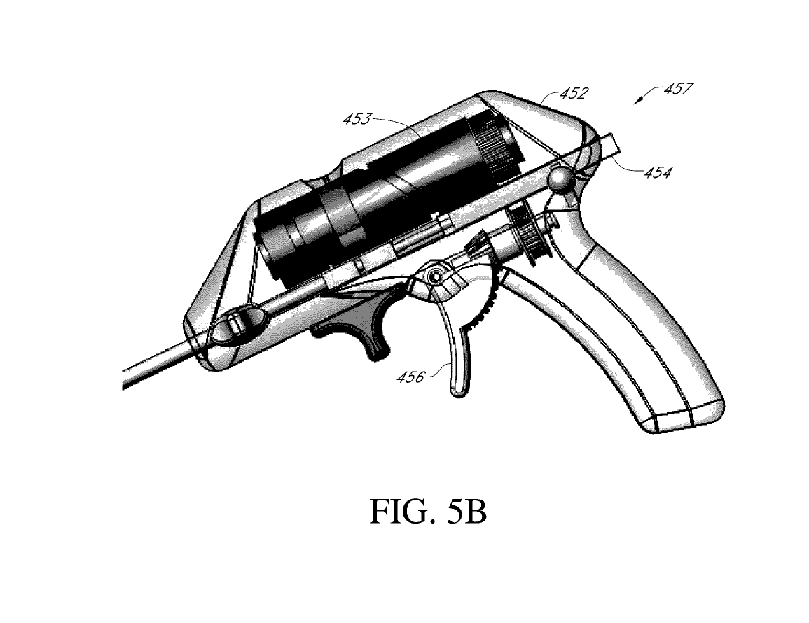

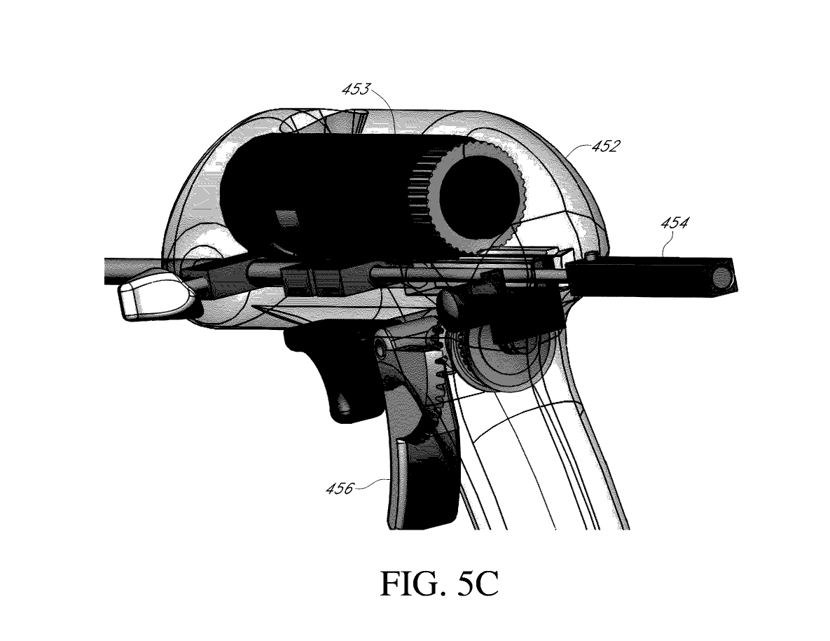

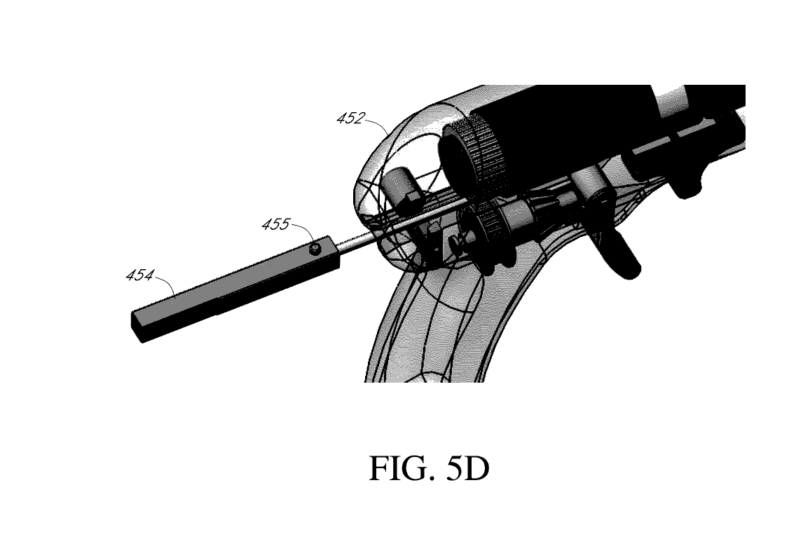

FIGS. 4 and 5A-D illustrate an example of a device for inverting diverticulum 415 that includes a swappable cartridge. FIG. 4 illustrates an exploded view of a device for inverting diverticulum 415 with swappable cartridge. FIG. 5A illustrates a side view of the device for inverting diverticulum 415 with swappable cartridge. FIGS. 5B-D illustrates a plurality of views of the handle of the device for inverting diverticulum 415 of FIG. 5A.

FIG. 4 illustrates an embodiment of a device for inverting diverticulum 415. In some examples, the device for inverting diverticulum 415 can include a handle 452. In some variants, the handle 452 can be attached to a plurality of components. As illustrated, the device for inverting diverticulum 415 can include a tissue tube 448, a basket shaft 422, a closer 420, a push rod 436, and a cartridge 454. Each of the components can be configured to be moveable relative to each other. As well, in some examples, each of the components can be coaxially disposed about each other.

In some examples, the device for inverting diverticulum 415 can include a tissue tube 448. During use, the tissue tube 448 can be located coaxially between the clip tube 428 and the basket shaft 422. The purpose of the tissue tube 448 is to support the tissue of the ostium of the inverted diverticulum when the clip tube 428 has not been inserted into the device for inverting diverticulum 415. In some examples, the tissue tube 448 can include castellated structures 450 at the distal end. As was discussed above, the purpose of the castellated structures 450 is to engage the closure clip 440 of the device for inverting diverticulum 415 and to provide the closure clip 440 with access to the tissue during treatment.

As with the other devices described above, the device for inverting diverticulum 415 can include a basket shaft 422. In some embodiments, the basket shaft 422 can be coaxially disposed about the tissue tube 448 such that the tissue tube 448 can be advanced or withdrawn relative to the tissue tube 448. In some examples, the basket shaft 422 can include a basket 424 located at the distal end of the basket shaft 422. The basket 424 of the basket shaft 422 can serve the same purpose as the baskets described above for the device for inverting diverticulum 115, 215, 315. As noted previously, the basket 424 can be used to gather the tissue of the ostium of the inverted diverticulum prior to clip placement.

In some embodiments, the device for inverting diverticulum 415 can include a closer 420 that is a tube that can be coaxially disposed about the basket shaft 422. In some examples, as described above for the device for inverting diverticulum 115, 215, 315, the closer 420 can be configured to advance distally about the proximal end of the basket 424. This can narrow the basket 424 as it is gathering the tissue of the ostium of the inverted diverticulum.

In some examples, the device for inverting diverticulum 415 can include a push rod 436 that can be advanced coaxially with the tissue tube 448, such that the tissue tube 448 is disposed about the push rod 436. In some embodiments, the push rod 436 can have an atraumatic tip 438 located on the distal end of the push rod 436. As discussed above, in some examples, the atraumatic tip 438 can be used to invert a diverticulum during treatment. In some examples, the push rod 436 can include a cartridge 454 that is configured to attach to a portion of the push rod 436. The cartridge 454 can be introduced into the handle 452 and can be advanced to the distal end of the device.

In order to provide a swappable closure clip 440, the device for inverting diverticulum 415 can be configured to include a clip tube 428 with a closure clip 440 attached to the distal end. In some embodiments, the cartridge 454 can be loaded with the closure clip 440 (and/or the clip tube 428) such that the closure clip 440 can be inserted into the device for inverting diverticulum 415 after the device for inverting diverticulum 415 has gathered the ostium tissue with the basket 424. As discussed above, this can provide the benefit of inserting a closure clip 440 after the size and/or shape of the ostium of the inverted diverticulum has been determined. As well, in some embodiments, the attachment of the closure clip 440 to the swappable cartridge 454 enables the user to deploy a plurality of closure clips 440 in the treatment of a plurality of diverticulum.

FIG. 5A illustrates a side view of the device for inverting diverticulum 415 with a cross-sectional view of the handle 452. As illustrated, the device for inverting diverticulum 415 can include a handle 452 on the proximal end 458 of the device for inverting diverticulum 415. The handle 452 can include a plurality of components that provide control of the distal end 457 of the device for inverting diverticulum 415 which engages and treats a diverticulum.

FIG. 5B-D illustrates a plurality of views of a handle 452 on a device for inverting diverticulum 415. In some embodiments, the 425 can include an actuator 456, a rotary cam drive 453, and an opening on the distal end 457 of the handle 452 that allows the insertion of the cartridge 454.

As illustrated, the cartridge 454 can be inserted into the distal end 457 of the handle 452. The cartridge 454 can include a rotary engagement 455 that is located on a surface of the cartridge 454. As the cartridge 454 is inserted, the rotary engagement 455 of the cartridge 454 can engage with a pathway located on a surface of the rotary cam drive 453. In some examples, the actuator 456 on the handle 452 can turn the rotary cam drive 453 when actuated. As the rotary cam drive 453 turns, it rotates the pathway located on the surface of the rotary cam drive 453 and advances the cartridge 454 in a distal direction. Once introduced, the cartridge 454 can engage with the handle 452 and enable continuation of an integrated closure clip 440 delivery process starting from the moment of introduction.

While the description generally refers to colonoscopies and treatments within a colon, the devices and methods described herein are not limited to applications within a colon. They can be used to invert and/or treat outpocketings (e.g., diverticula, aneurisms, etc.) in any body lumen. Any reference to a colonoscope should be understood to be applicable to endoscopes generally, and similarly, any reference to a colon should be understood to be applicable to any body lumen.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A clip placement device for diverticulum inversion, comprising:
a tubular body;
a clip attachment structure, wherein the clip attachment structure has a distal end that is attachable to a clip and wherein the clip attachment structure is configured to engage with the distal end of the tubular body; and
a pusher structure that is coaxially disposed within the tubular body, wherein the pusher structure is configured to removeably connect to the clip attachment structure.

2. A method of clip placement for diverticulum inversion, comprising:
positioning a distal end of a clip placement device along an outer wall of a colon at a diverticulum;
inverting the diverticulum into the lumen of the colon with the distal end of the clip placement device, wherein the clip placement device comprises a tubular body and a pusher structure that is coaxially disposed within the tubular body, and wherein the pusher structure is configured to attach to a clip attachment structure;
attaching the clip attachment structure to the pusher structure, wherein the clip attachment structure has a distal end that is attached to a clip;
advancing the pusher structure and the attached clip attachment structure along the tubular body until the clip protrudes from the distal end of the tubular body.

3. The method of claim 2, further comprising:
withdrawing the pusher structure with the attached clip attachment structure such that the pusher structure is withdrawn from the tubular body;
removing the attached clip attachment structure from the pusher structure;
attaching a second clip attachment structure to the pusher structure, wherein the second clip attachment structure has a distal end that is attached to a second clip; and
advancing the pusher structure and the attached second clip attachment structure along the tubular body until the second clip protrudes from the distal end of the tubular body.

4. The method of claim 2, wherein the clip attachment structure is configured to engage with the distal end of the tubular body.

5. A system for clip placement and diverticulum inversion, comprising:
a clip placement device comprising:
a tubular body,
a clip attachment structure, wherein the clip attachment structure has a distal end that is attached to a clip, and wherein the clip attachment structure is configured to engage with the distal end of the tubular body, and
a pusher structure that is coaxially disposed within the tubular body, wherein the pusher structure is configured to removeably connect to the clip attachment structure; and
a second clip attachment structure, wherein the second clip attachment structure has a distal end that is attached to a second clip, and wherein the second clip attachment structure is configured to engage with the distal end of the tubular body and is configured to removeably connect to the pusher structure.
